(19) United States
(12) Patent Application Publication
(10) Pub. No.: US 2017/0049701 A1
(43) Pub. Date: Feb. 23, 2017

(54) CLUSTOIDAL MULTILAMELLAR SOY LECITHIN PHOSPHOLIPID STRUCTURES FOR TRANSDERMAL, TRANSMUCOSAL, OR ORAL DELIVERY, IMPROVED INTESTINAL ABSORPTION, AND IMPROVED BIOAVAILABILITY OF NUTRIENTS

(71) Applicants: Steven M. Kushner, Oldsmar, FL (US); Bernard W. Downs, Lederach, PA (US)

(72) Inventors: Steven M. Kushner, Oldsmar, FL (US); Bernard W. Downs, Lederach, PA (US)

(21) Appl. No.: 15/228,884

(22) Filed: Aug. 4, 2016

Related U.S. Application Data
(60) Provisional application No. 62/201,225, filed on Aug. 5, 2015.

(51) Int. Cl. A61K 9/127; A61K 31/167; A61K 9/00; A61K 35/19

(52) U.S. Cl. CPC A61K 9/127; A61K 35/19; A61K 31/167; A61K 9/0053; A61K 9/0014; A61K 9/006; A61K 9/1278; A61K 2035/124

(57) ABSTRACT

Clustoidal multilamellar soy lecithin phospholipid structures are provided. A process enables comprehensive and uniform encapsulation of nutritional and/or pharmaceutical ingredients in multilamellar clustoidal soy lecithin phospholipid (prodosome) capsules facilitating superior absorption of nutritionally and pharmacologically active therapeutic substances that provide benefits following absorption of the energetically enhanced electrolyte-impregnated phospholipids. Methods of use for the soy lecithin phospholipid (SLP) materials are contemplated including delivery of one or more nutrients or nutritional/pharmaceutical compositions as desired through oral and topical administrations.

This application claims the benefit of earlier filed U.S. Provisional application No. 62/201,225, filed on Aug. 5, 2015, which is hereby incorporated by reference herein in its entirety for all purposes.

### TECHNICAL FIELD

A process enables comprehensive and uniform encapsulation of nutritional and/or pharmaceutical ingredients in electrolyte-impregnated high-phosphatidyl choline containing soy lecithin phospholipid capsules facilitating superior absorption of nutritionally and pharmacologically active therapeutic substances that provide benefits following absorption of the energetically enhanced phospholipids.

### BACKGROUND

In North America digestive malfunction in terms of disintegration, dissolution, and absorption of food and nutrients, is a widespread malady. Malabsorption is also an exacerbating factor in most chronic degenerative diseases that might benefit from dietary supplementation.

Effective digestion of food, food constituents, and dietary supplements is essential for maintaining overall health. The most prevalent health disorder in the North American culture is digestive problems. According to the Centers for Disease Control, after accidents, digestive disorders are the number one reason for emergency room visits in the United States (US Dept. of Health and Human Services, Centers for Disease Control and Prevention, National Center for Health Statistics. (National hospital ambulatory medical care survey: 2011)).

Prescription drugs and OTC products for digestive distress are among the most popular medications in the US, punctuating the prevalence and impact of this disorder. Digestive malfunction in terms of impaired disintegration, dissolution, and absorption of food and nutrients, is a widespread malady; it is also an exacerbating factor in most chronic degenerative diseases (CDD). As a consequence, widespread digestive problems impair the effective absorption of food constituents and the ability to benefit from food and dietary supplements. Translating this information into practical terms means that the most expensive dietary supplements are the ones that don't work because the body cannot properly disintegrate, dissolve and/or absorb their nutrient contents, essentially resulting in a colossal waste of money and progressive deterioration of health.

In 1985, the US natural products industry revenues (from all products and foods) totaled a little over $4 billion. As of 2012, that number has soared to over $137 billion. Such popular and widespread patronization of natural foods and dietary supplements would be expected to result in a reduction of the incidence of CDD. Yet, increasing rates of CDD provide evidence that the explosion of natural product purchases has not blunted the incidence of CDD even one iota. So, the popularity of natural products seems to have failed in improving the health of a nation and curtailing escalation in the incidence of CDD. However, an even greater indictment must be levied against the pharmaceutical industry with annual revenues in 2012 exceeding $331 billion. More money is spent on health issues in the US than any other country in the world. Yet even so, we have the highest rate of CDD. The focus has overwhelmingly been almost entirely on treatment in terms of symptom management and relief from immediate sufferings; the focus is not on reversing the cause of the suffering. Hence, the incidence of CDD continues to increase unabated while the juggernaut of treatment and healthcare costs continue to soar. This is a massive and glaring problem that demands a solution that medical technocracy is not equipped nor appropriate to handle.

Essentially, the ability of the body to achieve optimal functioning is dependent on the quality and absorbability of air, water, sunshine and food; the foundational resources from which the body is made and on which life is dependent. What is needed is a technology that enables nutrition, nutraceuticals, medical foods, and even pharmaceuticals that are orally ingested, topically applied, and/or delivered through other methods of entry into the body, to be effectively absorbed and become efficiently and effectively bioavailable to the body's tissues, strengthening and maintaining the optimal structure and function of every cell in the body or providing a pharmacological effect without depending on the competence of the digestive system's ability to disintegrate and dissolve its contents in order to be absorbed.

It is well-known that phospholipids are important molecules in biological systems. Cells are surrounded by a layer of phospholipids called the phospholipid bilayer (generally, “lipid bilayer”). This layer makes up your cellular and intracellular organelle membranes, forming a selectively permeable barrier, and is critical to a cell's ability to function. Phospholipids are arranged so that their water-repelling (hydrophobic) or ‘fat-loving’ tails are pointing inwards and their water-attracting (hydrophilic) heads are pointing outwards in this bilayer structure. This arrangement allows plasma membranes to be selectively permeable to dissolved substances such as proteins, ions and water. In biological systems, phospholipids allow cell membranes to be fluid. Their unique characteristics allow the cell membrane to be more malleable, taking different shapes and expanding or shrinking when necessary, such as when cells have to travel through very narrow capillaries in single file one at a time. Phospholipids also can act as signaling molecules for receptors inside and outside of cell surfaces, facilitating communications between cells. They can be split to produce secondary messengers in cellular systems. As a secondary messenger, phospholipids can signal for leukocytes to migrate to a site of infection, and they can also inhibit nerve cells when necessary.

Important Functions of Phospholipids
(1) Act as building blocks of the biological cell membranes in virtually all organisms.
(2) Participate in the transduction of biological signals across cell membranes.
(3) Act as efficient store of energy as with triglycerides.
(4) Play an important role in the transport of fat between gut and liver in mammalian digestion.
(5) Serve as an important source of acetylcholine which is the most commonly occurring neurotransmitter substance occurring in mammals.

One of the outcomes of a healthy diet combined with healthy digestion is the formation of liposomes from phospholipids in the diet. Owing to the diminished quality of the standard American diet, and the consequential widespread decline of digestive competence, the formation of liposomes in the gastrointestinal tract (GI) has been significantly compromised and diminished. Without the aid of the liposome, many of the nutrients would not otherwise adequately penetrate the epithelial wall of the intestines for eventual uptake into the cells. Liposomes are safe and important for facilitating optimal absorption of valuable nutrients. For example, naturally occurring liposomes are present in human breast milk (Koerner, M. M, et al., “Electrodynamics of lipid membrane interactions in the presence of zwitterionic buffers,” Biophysical 1 (2011) 101: 362-369). Liposome structures are biodegradable and biocompatible (‘body friendly’) enabling absorption through most tissues in the GI tract and alimentary tract from the mouth to the colon. In addition to water soluble vitamins, liposomes are beneficial for effective in situ delivery of fat-soluble vitamins, trace minerals, and naturally occurring phytonutrients including flavonoids, terpenes, and saponins (Keller, B. C., “Liposomes in nutrition,” Trends Food Sci. Techn. (2001) 12:25-31). However, conventional liposome technologies tend not to result in consistently uniform encapsulated finished products; tend to be unstable; and consequently can degrade rather rapidly but definitely over time. Moreover, unless encapsulating individual stand-alone ingredients, the composition and potencies of encapsulated ingredients can vary significantly and further result in encapsulation inconsistencies. These factors pose a major drawback to liposomal encapsulation.

Thus, if a way could be found to provide a stable, efficient delivery vehicle based on the advantages of liposomes, this would serve as a contribution to the medical and nutritional arts.

### SUMMARY

In one embodiment, the invention relates to an electrolyte-impregnated multilamellar clustoidal soy lecithin phospholipid (SLP) structure, also known as a prodosome.

In one embodiment, a process for making a multilamellar clustoidal soy lecithin phospholipid (SLP) structure is provided.

In one embodiment, a process for making one or more multilamellar clustoidal phospholipid structures comprises the steps of: (a) adding a naturally derived ionic mineral composition to water and mixing at high speed vortex to form ionically charged structured water; (b) adding phosphatidylcholine of at least 70% purity to the ion-treated water composition by mixing in a high speed vortex to form a liposomal mixture; (c) adding ethyl alcohol to the liposomal mixture by mixing in a high speed vortex to form the one or more multilamellar clustoidal phospholipid structures in water; and (d) allowing the multilamellar clustoidal phospholipid structures in water to cool to ambient temperature.

In one embodiment, a multilamellar clustoidal phospholipid vehicle for delivery of a cellular, subcellular, nutritional, nutritional, or pharmaceutical ingredient, comprises a solvent, phosphatidylcholine of at least 70% purity, and a naturally derived ionic mineral composition.

In one embodiment, a formulation for delivery of an active ingredient comprises the active ingredient encapsulated in a multilamellar clustoidal phospholipid vehicle, wherein the multilamellar clustoidal phospholipid vehicle comprises a solvent, phosphatidylcholine of at least 70% purity, and a naturally derived ionic mineral composition.

In one embodiment, a method for delivering an active ingredient to an individual comprises the steps of: (a) providing a formulation comprising the active ingredient encapsulated in a multilamellar clustoidal phospholipid vehicle, wherein the multilamellar clustoidal phospholipid vehicle comprises a solvent, phosphatidylcholine of at least 70% purity, and a naturally derived ionic mineral composition; (b) administering the formulation to the individual in need thereof.

### BRIEF DESCRIPTION OF THE DRAWINGS

FIG. 1 depicts, in one embodiment, a representative example from Group 1 (Subject #45) via transmucosal delivery, human subjects: (a) Baseline before intake of water; (b) Five minutes after taking 30 ml water; and (c) Five minutes after taking 30 ml VMP35 MNC.

FIG. 2 depicts, in one embodiment, a representative example from Group 2 (Subject #10): (a) Baseline blood test before the administration of VMP35 MNC; and (b) Five minutes after taking VMP35 MNC.

FIG. 3 depicts, in one embodiment, a representative example from Group 2 (Subject #11): (a) Baseline blood test before the administration of 30 ml VMP35 MNC; and (b) Five minutes after taking VMP35 MNC.

FIG. 4 depicts, in one embodiment, a representative example from Group 2 (Subject #40): (a) Baseline blood test before the administration of 30 ml VMP35 MNC; and (b) Five minutes after taking VMP35 MNC.

FIG. 5 depicts, in one embodiment, a representative example from Group 3 (Subject #49): (a) Baseline blood test before the administration of VMP35 MNC; (b) Five minutes after taking 30 ml VMP35MNC; and (c) Thirty minutes after taking 30 ml VMP35 MNC.

FIG. 6 depicts, in one embodiment, a representative example from Group 3 (Subject #49): (a) Baseline blood test before the administration of VMP35 MNC; (b) Five minutes after taking 30 ml VMP35MNC; and (c) Thirty minutes after taking 30 ml VMP35 MNC.

FIG. 7 depicts, in one embodiment, confocal microscopic imaging showing CD42b stained platelets at 4 hours post exposure to the PEHPs. (The dots highlighted by a black arrow within a white circle are the stained platelets.)

FIG. 8 depicts, in one embodiment, a confocal microscopic imaging showing CD42b stained platelets at 24 hours post exposure to the PEHPs. (The dots highlighted by black arrows within a white circle are the stained platelets.)

FIG. 9 depicts, in one embodiment, cytokine and growth factor analysis (Fibrin, IL-6 and IL-8) following platelet exposure of Epiderm™ tissue layer for 24 hours. (No treatment=untreated controls)

FIG. 10 depicts, in one embodiment, cytokine and growth factor analysis (IL-1β, MCP5, and VEGF) following platelet exposure of Epiderm™ tissue layer for 24 hours. (No treatment=untreated controls).

FIG. 11 depicts, in one embodiment, histology of control tissues (untreated and prodosome vehicle control) at 4 hours.

FIG. 12 depicts, in one embodiment, histology of PEHPs exposed tissues at 4 hours.

FIG. 13 depicts, in one embodiment, histology of untreated and vehicle control tissues at 24 hours.

FIG. 14 depicts, in one embodiment, histology of PEHPs exposed tissues at 24 hours.

FIG. 15 depicts, in one embodiment, histology of PEL (lidocaine) exposed tissues at 24 hours.

FIG. 16 shows, in one embodiment, components of the trace mineral concentrate.

### DETAILED DESCRIPTION

### Definitions

As used herein, the term “clustoid(s)” refers to clusters of liposomal spheres. Further, “multilamellar clustoidal’ refers to clusters of liposomal spheres within a liposomal sphere and clusters of those liposomal spheres within a liposomal sphere, etc., up to hundreds of concentric layers.

As used herein, the term “prodosome” refers to the “energetically enhanced (EFIquence-treated) liposome that comprises the complex of multilamellar clustoidal liposomal structures.” Specifically, prodosome refers to electrolyte (ion)-impregnated phospholipid liposome complex having multilamellar clustoidal liposomal structures.

The term “liposome” as used herein means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. In order to cross intact mammalian skin, lipid vesicles must pass through a series of fine pores, each with a diameter less than 50 nm, under the influence of a suitable transdermal gradient. Therefore, it is desirable to use a liposome which is highly deformable and able to pass through such fine pores.

The term “bioavailability” refers to a measurement of that portion of an administered drug which reaches the circulatory system (e.g. blood, especially blood plasma) when a particular mode of administration is used to deliver the drug. Enhanced bioavailability refers to a particular mode of administration's ability to deliver nutrients, including oligonucleotides, nutraceutical particles, and drugs to the peripheral blood plasma of a subject in need relative to another mode of administration. For example, when a non-parenteral mode of administration (e.g. an oral mode) is used to introduce the drug into a subject in need, the bioavailability for that mode of administration may be compared to a different mode of administration, e.g. bioavailability correlates with therapeutic efficacy when a compound's therapeutic efficacy is related to the blood concentration achieved.

Prodosomes

While the liposome is naturally a zwitterion molecule, the inclusion of the mineral ions in a similar proportion that exists in human blood, within every portion of the present complex of liposomal clustoidal spheres creates previously non-existent electrical properties of the liposomes (called “prodosomes”). Based on electrostatic properties, mineral ions incorporated into the water used for creating liposomes become part of the liposome structure itself; resembling the ionic proportions that exist in human blood, for example. This enhances the ability of the liposomal transport sphere to transport and facilitate encapsulated nutrient absorption. This is in addition to encapsulating supplemental minerals of a nutrient formula containing one or more nutrient components within their liposomal spherical structures as a nutrient or nutritional payload.

In one embodiment, the solar-dried electrolyte source material being infused into the phospholipids is ionic in nature. This property infuses the ions into the manufactured liposomes and creates electrical/energetic/frequency properties of the phospholipid-based liposomal structures. The liposome (prodosome), in essence, becomes a dynamically charged compound, resembling more of a biological material, with similar ionic amounts as exist in human blood, with greater bio-functionality and potential for transport and delivery of nutrients, and contributing beneficial biological activities on their own.

Electrolytes are also important intracellular pH buffers. Following the depletion of intracellular electrolytes and exhaustion of other primary buffers, hemoglobin is expended to maintain intracellular pH. This changes not only the oxygen carrying abilities of the hemoglobin but also polarity (negative ion concentration), and results in excessive red blood cell aggregation. Improvement in red blood cell morphology and plasma rheology, for example, are evidence of improvements in blood viscosity, negative ion concentration, pH, and blood functionality, i.e. oxygenating and hydrating properties.

With the liposome (prodosome) now infused and saturated with a comprehensive range of naturally occurring energetically active ions, there is a greater potential that the entire multilamellar clustoidal structure may act as a pH buffering agent for the tissues. It is likely that there is a re-balancing of pH in tissues where the liposome releases its payload as well as when the liposomal membranes sequentially begin to degrade and release their bioactive ions. This re-balancing of pH and restoration of optimal ionic properties will foster a more advantageous environment for nutrient utilization. As pH rebalances, healthy blood morphology, rheology and hematology (i.e. viscosity, form, structure, oxygenation, hydration, etc.) are restored.

Phospholipids have an adhesive property owing to the hydrophilic and hydrophobic properties of the molecule. As a result, a natural tendency of a phosphatidyl choline-based liposome is its ability to adhere to tissues, especially the mucosa of the GI tract. This attribute promotes transmucosal nutrient transport from the sublingual tissues in the mouth to the tissues of the intestine. Prolonged adherence of the liposome to the surface of the villi and microvilli translates to a longer portion of time that nutrients can diffuse across the membranes into the blood stream. More importantly, the extended time that the liposome remains attached to the mucosal membrane gives additional time for the mineral ions to saturate the same membranes. Moreover, the lipid bilayer construction created by phospholipids is readily incorporated into the cell membrane phospholipid bilayer. By continually saturating the junctions where nutrients are absorbed, an advantage is afforded for more complete nutrient transport. This is due to mineral ions' contribution to maintaining the osmotic gradient in the lipid bilayer of cell membranes that facilitate nutrient diffusion and maintain electroneutrality.

The fact that the liposome remains attached to the mucous membrane for longer periods of time means the mineral ions remain there as well. Again, this means that there is a longer period of time where nutrient exchange, facilitated by cellular ions, can be carried out. Moreover the rationale of infusing the mineral ions within the entirety of the liposome is borne out by the fact that as each layer of the multi-lamellar sphere degrades and releases its nutrients in the GI mucosa, there is a simultaneous and consistent release of mineral ions as a result of liposomal (prodosome) degradation. This is as opposed to bound minerals just being present within the sphere of a “simple” liposome that can release at a single instant and then must be absorbed into the bloodstream. Our novel process enables mineral ions to be available throughout the entire process where each successive layer of phospholipids and their nutrient contents, both fat and water-soluble, are being released from the disintegrating spheres, along with phospholipid-infused ions, and made available for diffusion and bioactivity.

Perhaps one of the more interesting aspects of this technology is its ability to increase Zeta Potential within the liposome (prodosome) itself and consequently in surrounding fluids where the liposome degrades and releases its nutritional payload, including its free ions. Zeta potential is defined as the electrical potential of dispersed particles in colloidal solutions. The higher the Zeta Potential, the greater the dispersion and subsequent stability of the solution. A higher Zeta Potential indicates a stronger level of electrostatic repulsion within the solution. (This creates a more stable liposome, a key factor in maintaining the biologically active properties and efficacy of a nutritional compound or nutrient formula.) This not only holds true for the solution (in this case the liposomal concentrate) but also this potential can be transferred to the surrounding tissues as the liposome disintegrates/degrades. The electrostatic repulsion and separation of biological materials (i.e. erythrocytes, leukocytes, platelets, etc.) is exactly the environment that is desirable within the bloodstream of a human subject, for example.

This type of environment helps to ensure adequate red blood cell circulation and therefore oxygenation over a larger surface area. The opposite consequence of this would be aggregation (undesirable) and less free-flowing red blood cells. Therefore, part of the novelty of the process technology as described herein is in creating a transfer of Zeta potential through the direct action of the liposome itself as it enters into surrounding plasma. As the Zeta potential is increased in the surrounding blood, allowing for better circulation of red blood cells, the overall rheology of the blood is improved, thereby allowing for a greater flow of the nutrient payload that has been delivered by the multilamellar clustoidal liposome structures. Research has shown that both Zeta potential and particle size within a colloidal solution can be modified by the inclusion of an ionic species, for example, in the present preparation of clustoidal multi-lamellar SLP structures (prodosomes).

Recent research has also shown that varying degrees of vortex speed can decrease particle size in a colloidal solution while simultaneously increasing Zeta potential. This also serves to allow the present liposomal technology to increase its surface area coverage. This is because the present process includes using high-speed RPMs within small mixing containers. This condition allows the mineral ions to more thoroughly disperse in a more uniform manner within the phospholipid matrix, which directly leads to a higher Zeta potential. Moreover, typical Zeta potential has to do with an electrokinetic potential between the surface of the colloidal particle and any point in the mass of the liquid medium. Without being bound by any theory, it is believed that because the present process involves increasing ionic concentrations within the water prior to the production of the multi-lamellar liposome, multiple surfaces are generated surrounded by multiple liquid mediums, into which the active substrate can permeate the inter-phospholipid molecular spaces or interstitial lumens. Thus, the multi-lamellar SLP structure is produced. In addition, it appears evident that we have created a Zeta potential within the multitude of layers of this clustered multi-lamellar liposomal sphere, called a prodosome. Consequently a unique aspect of this technology is that as the liposome dissolves sequentially layer by layer, positive benefits of the increased Zeta potential from each surface layer is conferred into the surrounding medium into which the liposome dissolves, in this case the sublingual mucosa (alimentary) and small intestine (GI), facilitating rapid and prolonged absorption into the bloodstream.

The absorption of food and most supplemental minerals primarily takes place within the small intestines, although ionic minerals can be absorbed through the sublingual mucosa. As food matter passes through the intestines, minerals transfer into the blood stream through the walls of the intestines by way of the villi. This can only happen if the minerals are in an ionic form. When the stomach is functioning properly, stomach acid normally ionizes minerals in foods and supplements. But, this only happens when the stomach is functioning properly, which, according to statistics mentioned above, is not commonplace in North America. Most mineral supplements contain bonded minerals (e.g., calcium carbonate, magnesium oxide, etc.) that must be ionized for optimal absorption and utilization in the body.

In one aspect, encapsulating nutritional, nutraceutical, or pharmaceutical substrate(s) in soy lecithin phospholipid (“SLP”) capsules enables superior absorption of nutritionally and pharmacologically therapeutic substances. This disclosure offers significant therapeutic health benefits due to its energetically enhanced phospholipid properties impacting delivery of nutrients and/or drugs, including, but not limited to: 1) neuroprotection, regulation of brain activity, improved memory and resistance to stress, reduced depression risk, and mitigation of the progression of neurodegenerative diseases like ALS, MS, Alzheimer's and Parkinson's disease; 2) positive influences on cellular growth, development, and energy generation due to participation in molecular transport, and cellular organelle and intracellular organelle structure and function; 3) acceleration of tissue and organism regeneration after trauma, damage, illness, and/or physical exertion, including wound healing; 4) limiting cholesterol absorption from the gastrointestinal tract; 5) beneficial outcomes in liver therapy (steatosis, alcohol intoxication, etc.); 6) inhibition of inflammation factors, some of which are pathogens of the alimentary canal and cancer promoters (e.g. of colon and adenoma; Keller, B. C., “Liposomes in nutrition,” Trends Food Sci. Techn. (2001) 12:25-31); and (7) immune support.

More specifically, the current disclosure is directed to a clustoidal multi-lamellar phospholipid based material (“prodosome”) that is infused and fortified with an electrolyte mineral complex comprising more than 70 naturally occurring macro- and trace minerals in ionic form. Trace minerals are naturally occurring minerals derived from evaporated inland sea water in an ionic form. Trace minerals include, but are not limited to, iron ion, copper ion, zinc ion, manganese ion, selenium ion, chromium ion, iodine ion, and boron ion. Macro minerals include, but are not limited to calcium ion, magnesium ion, phosphorous ion, potassium ion, chloride ion, and sulfur ion. The final material possesses an electrical potential structurally integrated into the SLP sphere at a micron/nano level. The SLP sphere in this form has now become more than just a transport compartment, but also possesses its own unexpected beneficial functionality that facilitates improved utilization of nutrients encapsulated within the SLP liposomal spheres. The present invention provides a myriad of electrolytic materials, simultaneously with encapsulated nutrients, that contribute to and govern cellular fluid balance and therefore are instrumental in all metabolic processes including cellular exchange of nutrients and waste removal.

Existing liposome technologies use lecithin and, those of higher quality, use phosphatidyl choline for the most part. Regardless of the phospholipid-source material, these technologies are generally mixed in a stereotypical fashion with no other additives or compounds utilized within the source material(s). The result is generally a relatively unstable product that degrades of its own accord in a relatively short period of time due to variations in temperature; agitation; composition of the substrate; interaction of the phospholipids with the encapsulated substrate; and pH, among other factors, that can result in agglomeration leading to degradation and delineation of the phospholipid bilayer membrane of the multilamellar spheres.

Biological Capacitor

The prodosome technology as described herein creates clusters of multilamellar liposomal structures in concentric layers of activated ion-infused liposomes within a liposome; and the multilamellar clusters of those molecules within an activated ion-infused liposome; up to hundreds of concentric layers, described as multilamellar liposomal clustoids, now called SK713 SLP Prodosomes, or ‘Prodosomes’. In addition to protecting the nutritional contents, complex multilamellar clustoidal structures (the ‘SK713 SLP’ complex) effectively function as biological capacitors, containing and confining the biochemical and/or energetic potential of the ion-infused (energy frequency imprinting) phospholipids. However, this biological capacitor function would not occur in normal liposomes (see Table 1) and is only possible because of the energy frequency imprinting (a.k.a. ‘EFIquence’ technology, available from Victory Nutrition International, Lederach, Pa.) of the SK713 SLP process technology.

One objective in an embodiment of this invention is to supply already naturally ionized minerals that can be fully absorbed in vivo. The Energy Frequency Imprinting (trading as EFIquence™ Technology) process infuses and saturates the phospholipids with a full spectrum of solar-dried ionic minerals from ancient sea beds that supply minerals in biocompatible amounts and a proportion to the blood.

The electrolytes within the phospholipid matrix of the present invention are in ionic form; the most natural state where they are naturally charged, biologically active minerals that are bioavailable and soluble in water. This material is derived from the Great Salt Lake, then solar-dried, and containing over 72 ionic minerals that are about eight to ten times more concentrated than regular seawater and significantly more concentrated than colloidal minerals. Colloidal minerals are of larger particles size and contain no ionic charge as compared to the trace minerals used in this invention. In addition, the ions contained in the Prodosomes are at a similar percentage volume to that exists in human blood.

As described herein, the biological capacitor function of the multilamellar SLP clustoids (Table 2) would not occur in normal liposomes (Table 1) and is only possible because of the energy frequency imprinting (a.k.a. ‘EFIquence’ technology, available from Victory Nutrition International, Lederach, Pa.) of the SK713 SLP process technology. The novel multilamellar clustoidal phospholipid encapsulation technology of the present invention (SK713 SLP/“Prodosomes”) was developed to facilitate more stable, competent and comprehensive synchronized absorption and synchronized bioavailability and bioactivity of orally ingested nutrition. The SK713 SLP is distinctly unique and superior to any previous liposomal technologies and, unlike previous versions: contains more phospholipid substrate, which is impregnated and saturated with solar dried electrolytes in an ionic state; is demonstrably and significantly more stable; and is consistently more uniform and shown to be more efficacious for nutrient delivery than other liposomal technologies tested. Moreover, the ion-infused SK713 SLP makes a nutritional contribution to improving the structure and function of inter- and intracellular membranes and molecules.

TABLE 1
Electrical Resistance of Normal Liposome Solutions (Reference)

| Sample #1 Pure | 1000X Setting |
|---|---|
| Liposome | (in Ohms) |
| Distilled Water = 50 ml. | 600 |
| Drops 1 | 400 |
| Drops 2 | 380 |
| Drops 3 | 380 |
| Drops 4 | 360 |
| Drops 5 | 350 |
| Drops 6 | 300 |
| Drops 7 | 300 |
| Drops 8 | 300 |
| Drops 9 | 300 |
| Drops 10 | 280 |
| Drops 11 | 280 |
| Drops 12 | 280 |
| Drops 13 | 280 |
| Drops 14 | 280 |
| Drops 15 | 280 |
| Drops 16 | 280 |
| Drops 17 | 280 |
| Drops 18 | 280 |
| Drops 19 | 260 |
| Drops 20 | 260 |
| Drops 21 | 260 |
| Drops 22 | 260 |
| Drops 23 | 260 |
| Drops 24 | 240 |
| Drops 25 | 240 |
| Drops 26 | 220 |
| Drops 27 | 220 |
| Drops 28 | 220 |
| Drops 29 | 220 |
| Drops 30 | 200 |
| Drops 40 | 160 |
| Drops 50 | 150 |
| Drops 60 | 150 |
| Drops 70 | 140 |
| Drops 80 | 135 |
| Drops 90 | 125 |
| Drops 100 | 115 |

TABLE 2
Biological Capacitor Function of the Multilamellar SLP Clustoids

| Sample #2 | | | | |
|---|---|---|---|---|
| Prodosome | | 100X | 1000X | |
| (Multi- | 1000X Setting | Setting | Setting | 100X Setting |
| lamellar SLP) | (in Ohms) | (in Ohms) | (in Ohms) | (in Ohms) |
| Distilled Water = | 600 | | 600 | |
| 50 ml. | | | | |
| Drops 1 | 350 | | 350 | |
| Drops 2 | 220 | | 220 | |
| Drops 3 | 160 | | 160 | |
| Drops 4 | 140 | | 150 | |
| Drops 5 | 140 | | 140 | |
| Drops 6 | 120 | | 120 | |
| Drops 7 | 120 | | 120 | |
| Drops 8 | 120 | | 100 | |
| Drops 9 | 100 | | 90 | |
| Drops 10 | 90 | | 80 | |
| Drops 11 | 80 | | 80 | |
| Drops 12 | 80 | | 70 | |
| Drops 13 | 70 | | 66 | |
| Drops 14 | 70 | | 64 | |
| Drops 15 | 65 | | 62 | |
| Drops 16 | 65 | | 58 | |
| Drops 17 | 60 | | 56 | |
| Drops 18 | 60 | | 56 | |
| Drops 19 | 56 | | 54 | |
| Drops 20 | 54 | | 54 | |
| Drops 21 | 54 | | 52 | |
| Drops 22 | 52 | | 50 | |
| Drops 23 | 50 | | 50 | |
| Drops 24 | 50 | | 48 | |
| Drops 25 | 50 | | 48 | |
| Drops 26 | 48 | | 48 | |
| Drops 27 | 48 | | 46 | |
| Drops 28 | 46 | | 44 | |
| Drops 29 | 46 | | 44 | |
| Drops 30 | 45 | 300 | 42 | 280 |
| Drops 40 | 38 | 220 | 32 | 200 |
| Drops 50 | 28 | 180 | 28 | 160 |
| Drops 60 | 28 | 170 | 26 | 150 |
| Drops 70 | 26 | 150 | 26 | 140 |
| Drops 80 | 24 | 140 | 24 | 130 |
| Drops 90 | 23 | 125 | 24 | 120 |
| Drops 100 | 22 | 120 | 22 | 120 |

As shown in the comparison of Tables 1 and 2, testing was performed to determine the difference in electrical resistance between distilled water, a basic liposome dissolved in distilled water, and the SK713 SLP Prodosomes dissolved in distilled water, and the trace mineral concentrate in pure form that is used in the processing of the prodosomes. A standard multi-meter (Armaco Brand 20A) was used and was set to measure ohms with a digital output. Ohms are a measurement of the electrical resistance that can be found in a particular solution or compound. Tests were run on both the 100× and 1000× setting, the 1000× setting being more sensitive to ionization. Pure distilled water was used as a control, and as the medium for dissolving the various liquids to be tested. All materials including the distilled water were allowed to reach room temperature. The amount of water used in each test was a volume of 50 ml and all came from a single bottle. All containers used for testing were glass. In all cases, each material to be tested was added 1 drop at a time into the water and the multimeter was used to detect resistance as determined by Ohm readings. After the initial tests were completed, identical testing was repeated to ensure uniformity of results.

In measuring pure distilled water, the detection of ohms, as shown on the digital readout at the 100× setting was not detectable indicating infinite resistance and therefore no conductivity. At the 1000× setting the reading was 600 (Table 1).

Next, the basic liposome was added 1 drop at a time to 50 ml of distilled water with an Ohm reading being taken after each drop was manually stirred in the water (Table 1). On the 100× setting, there was no evidence through the multimeter readings to show any Ohms, and therefore no electrical conductivity, even up to 100 drops of the liposome solution in the water medium, confirming the electroneutrality of the phospholipid molecules. At the 1000× setting, 1 drop lowered the resistance from the 600 level to 400, 2 drops only changed the reading to 380, the same with 3 drops, while 4 drops of liposome lowered only to 360, and 5 drops to 350. At 6-9 drops the reading was maintained at 300 Ohms (Table 1).

Next, the SK713 SLP Prodosome was added 1 drop at a time to 50 ml of distilled water with an Ohm reading being taken after each drop was manually stirred in the water (Table 2). At the 1000× setting, 1 drop lowered the resistance from the 600 level to 350, 2 drops changed the reading to 220, the same with 3 drops lowering the resistance level to 220. Four drops of Prodosome decreased the reading to 140, and stayed the same at 5 drops. At 6-8 drops, the reading was maintained at 120 while the 9th drop of Prodosome lowered the Ohms to 100. After the second drop of Prodosome was added to the water medium and tested, and as subsequent tests were performed, the decrease in resistance and concurrent increase in conductivity over the basic liposome was approximately 3 times as great. Additionally, in comparing the Ohms reading of the basic liposome to the Prodosome testing after each drop 10-30, the increase in conductivity of the Prodosome material was consistently 3-4 times more than the basic liposome. Also, with the basic liposome being added up to 100 drops in the water, there was no evidence of lessening resistance and therefore no conductivity at the 100× setting. On the other hand, the Prodosome test done with the 100× setting on the multimeter did begin to show a lessening of resistance according to Ohms at drop number 30 and continued to gradually decrease in resistance at testing of drops 30-100.

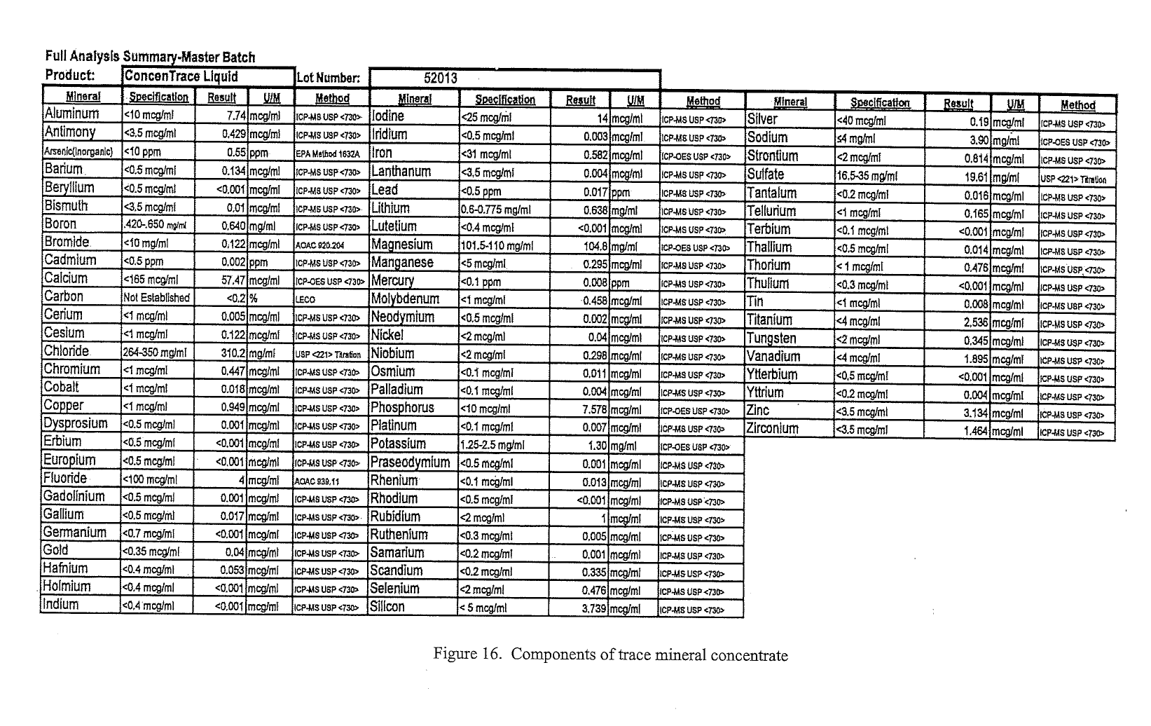

Finally, the pure trace mineral concentrate (TMC) used in the production of the Prodosome was added to the distilled water in an amount equivalent to that found in the same volume of Prodosome (Table 3). In other words, the TMC was added to a pre-measured quantity of water at a fraction of the total volume of Prodosome so as to ensure the amount of TMC would be the same as exists in the Prodosomes at each measurement, drop for drop, comparing the Prodosome to the TMC. In this test, the ionization was strong enough to only require the multimeter to be used at the 100× setting. At 1 drop of the TMC, the reading was 1 drop 500, 2 drops 280, 3 drops 200, 4 drops 160, 5 drops 140, 6 drops 120, and drops 7 and 8 at 100. The minerals contained in the TMC are listed in FIG. 16.

TABLE 3
Electrical Resistance of Solutions Containing Pure Ionic Trace Minerals

| Sample #1 Pure | 100X Setting |
|---|---|
| Ionic Trace Minerals | (in Ohms) |
| Drops 1 | 500 |
| Drops 2 | 280 |
| Drops 3 | 200 |
| Drops 4 | 160 |
| Drops 5 | 140 |
| Drops 6 | 120 |
| Drops 7 | 100 |
| Drops 8 | 100 |
| Drops 9 | 80 |
| Drops 10 | 80 |
| Drops 11 | 75 |
| Drops 12 | 75 |
| Drops 13 | 65 |
| Drops 14 | 65 |
| Drops 15 | 65 |
| Drops 16 | 65 |
| Drops 17 | 65 |
| Drops 18 | 65 |
| Drops 19 | 65 |
| Drops 20 | 60 |
| Drops 21 | 45 |
| Drops 22 | 40 |
| Drops 23 | 38 |

The readings of the multimeter in Ohms for the Prodosomes versus the TMC were consistently less by an order of magnitude (10×), drop for drop. Again, while there was an order of magnitude greater drop in resistance from the TMC, the concentration of ions in both the TMC/water mixture and the Prodosomes/water mixture was the same. This suggests strongly that the Prodosome material is acting as an effective insulator (‘biological capacitor’), and is evidence of the electrical activity showing in the Prodosome material only coming from the ionic minerals contained on the outermost phospholipid layer of the Prodosome clustoidal sphere. Being neutral, the distilled water medium does not allow the Prodosome sphere to completely disintegrate, therefore the balance of the ionic material would be contained, or insulated, in the lower levels of the multi-lamellar clustoidal spheres. This would also promote the benefits of the conductivity supplied by the release of the infused ionic TMC to be sustained over an extended period of time, as each layer of the multi-lamellar clustoidal Prodosome sphere sequentially disintegrates in the more alkaline environments of the body (i.e. mouth, intestine and possibly blood).

The biological capacitor function of the multilamellar SK713 SLP clustoids would not occur in normal liposomes (see Table 1) and is only possible because of the energy frequency imprinting (a.k.a. ‘EFIquence’ technology) of the SK713 SLP process technology. The novel multilamellar clustoidal phospholipid encapsulation technology of this present invention (SK713 SLP/Prodosomes) was developed to facilitate more stable, competent and comprehensive synchronized absorption and synchronized bioavailability and bioactivity of orally ingested nutrition. The SK713 SLP is distinctly unique and superior to any previous liposomal technologies and, unlike previous versions, contains more phospholipid substrate, which is impregnated and saturated with solar dried electrolytes in an ionic state; is demonstrably and significantly more stable; and is consistently more uniform and shown to be more efficacious than other liposomal technologies tested.

Clustoidal Multilamellar SLP Encapsulated Nutraceutical Multivitamin Formulations (SK713 SLP Encapsulated VMP35 Multinutrient Complex)

In one embodiment of the invention, the prodosomes based multivitamin formulation induced a beneficial effect on the properties of human blood by promoting rapid delivery of their nutritional contents to a human subject in vivo. This embodiment relates to a novel clustoidal multilamellar soy-lecithin-phospholipid encapsulation formulation (“SK713 SLP Encapsulated VMP35 Multinutrient Complex” or “VMP35 MNC”), which comprises, among other ingredients, multivatitavamins, such as vitamins A, C, D3, E, B1, B2, B3, B6, and B12. The formulation was designed to be administered transmucosally. The components of VMP35 MNC Formulation are described in the Table 4. However, the transmucosal route of administration of this formulation was not intended to be limiting. As understood by a person skilled in the art, the studied multivatimin formulation is also suitable for other routes of oral administration. Testing results showed that VMP35 MNC is a superior nutraceutical supplement that is able to effect positive changes in morphological, hematological, and rheological properties of human blood, and to overcome the limitations of those with various underlying digestive inefficiencies (Shoji, Y., et al., “Nutraceutics and delivery systems,” J. Drug Target (2004) 12:385-391).

TABLE 4
SK713 SLP Encapsulated VMP35 Multivitamin,
Mineral & Phytonutrient Formulation

| | | Unit of |
|---|---|---|
| INGREDIENT | Per Serving | Measure |
| R/O water | 26300 | mg |
| Vitamin A (Retinyl Palmitate) | 5000 | IU |
| Vitamin C (Ascorbic acid) | 60 | mg |
| Vitamin D3 (Cholecalciferol) | 0.025 | mg |
| Vitamin E (Alpha-tocopheryl Succinate) | 15 | IU |
| Vitamin B1 (Thiamin HCl) | 1.5 | mg |
| Vitamin B2 (Riboflavin) | 1.7 | mg |
| Vitamin B3 (Niacin) | 20 | mg |
| Vitamin B6 (Pyridoxine HCl) | 2 | mg |
| Folic acid | 400 | mcg |
| Vitamin B12 (Cyanocobalamin) | 5 | mcg |
| Biotin | 300 | mcg |
| Pantothenic acid (d-calcium pantothenate) | 10 | mg |
| Calcium lactate | 100 | mg |
| Iodine (potassium iodide) | 0.15 | mg |
| Magnesium citrate | 100 | mg |
| Zinc sulfate | 10 | mg |
| Sodium selenite | 0.07 | mg |
| Copper gluconate | 1 | mg |
| Manganese sulfate | 2 | mg |
| Chromium chloride | 0.12 | mg |
| Potassium citrate | 99 | mg |
| Choline bitartrate | 20 | mg |
| Inositol | 20 | mg |
| White pine cone extract | 5 | mg |
| BiAloe Concentrated 200:1 Water Extract | 20 | mg |
| VMP35 1:1 Herbal Blend: | 1700 | mg |
| Astragalus Root extract 1:1-247.5 mg | | |
| Ginger Root extract 1:1-99.95 mg | | |
| Green tea Leaf extract 1:1-199.92 mg | | |
| Fo ti Root extract 1:1-199.92 mg | | |
| Hawthorne berry extract 1:1-150.96 mg | | |
| Elderberry extract 1:1-99.95 mg | | |
| Eluthero Root extract 1:1-150.96 mg | | |
| Chamomile Flower extract 1:1-199.92 mg | | |
| Citrus bioflavonoids (from rose | | |
| hips) 1:1-199.92 mg | | |
| Gotu kola Leaf extract 1:1-150.96 mg | | |
| SK713 SLP | 342 | mg |

One of the major components of VMP35 MNC formulation is a specially prepared high grade soy lecithin material that contains a minimum of 85% phosphatidylcholine (>85PC), an essential phospholipid. While most lecithin products contain only 19-21% PC (Scholfield, C. R., “Composition of soybean lecithin,” J. Amer. Oil Chem. Soc. (1981) 58:889-892). The high PC content in SK713 SLP ensures thorough formation of liposomes. In addition to acting as biological capacitors and protecting the nutritional contents, multilamellar liposome phospholipids offer several health-related benefits. Due to their role in molecular transport, phospholipids also influence cell growth and development, and speed up organism regeneration after physical exertion. They limit cholesterol absorption from the gastrointestinal tract and are beneficial in liver therapy, for instance, in the treatment of steatosis. Phospholipids inhibit inflammation factors, some of which are pathogens of the alimentary canal and promoters of cancers, for example, adenoma, and colon cancer (Ambroziak, A., et al., “Milk phospholipids as nutraceutic,” Pol. Merkur. Lekarski. (2013)34:62-66).

The multi-lamellar or multisphered-multilayered-clustoidal structure of SK713 SLP, unlike standard liposome technology, is capable of encapsulating a diverse range of nutrients simultaneously. Through experimentation SK713 SLP was found to form vesicles made up of hundreds of concentric lipid bilayers that range in size from 100 nanometers to 500 micrometers and are made up of a few dozen to several thousand molecules (Keller, B. C., “Liposomes in nutrition”, Trends in Food Sci Techn (2001) 12:25-31). As soon as the concentration of phospholipids reaches critical mass, the water-repelling ends organize to form the liposomes with the lipophilic (fat-attracting) hydrocarbon chains oriented inwards and the hydrophilic (water-attracting) groups facing outwards, forming the lipid bilayer structure.

The SK713 SLP multilamellar liposomes form spontaneously as the electrostatic and adsorptive properties lower surface tension (surfactant). The net result is thorough and complete phospholipid encapsulation (or entrapment) of nutritional ingredients within multiple layers of nano to low micrometer sized spheres. This electrostatic encapsulation is effective for encapsulating and transporting both water and fat-soluble nutritional ingredients including phytonutrients within the same spherical structure (Akbarzadeh, A. et al., “Classification, preparation, and applications”, Nanoscale Res. Lett. 2013 8:102; Helfrich, W., “Distributions of vesicles: The role of the effective rigidity of membranes,” J. Phys. (1984) 47(2): 321-329).

a. Encapsulation of Nutrients

One of the limitations of encapsulating nutrients within the SLP transport spheres is the relative insolubility of some ingredients in water. Many nutritional compounds, especially inorganic minerals and resinous phytonutrients, are not readily soluble in water. To overcome this obstacle, prior to SK713 SLP processing, all materials are pre-processed in a low sheer tri-blender using jet-compression-particle-processing technology. This step is akin to a wet-milling process. In essence, the nutritional/nutraceutical materials are added directly to distilled water. The admixture is then blended at a low and consistent speed for a specific time, depending on the viscosity of the liquid and the physical and chemical properties of the added components. At the same time, water is circulated to create a secondary motion. No excess heat is produced in the mixing process. The low heat production combined with low shear used in the mixing step preserves the physicochemical stability of the nutrients and botanicals contained within the solution or suspension. The process continues for a period of time to substantially reduce particle size and to achieve consistency and uniformity of the mixed materials over successive batches. The electrolyte-impregnated SK713 SLP compound is then added to encapsulate these nutraceutical particles with greatly reduced particle size. Importantly, this preparation greatly improves bioavailability of the nutrients and botanicals. This preparation further ensures that previously insoluble materials can now be blended and dispersed into a semisolid or even a liquid state. The liquid concentrate is made up of the high-grade lecithin (>85% PC) combined with an amount of alcohol in exact proportions and blended at specific speeds for a specified time to achieve a solution with the right consistency, viscosity, and grade of material. The SK713 SLP material can then be blended into the liquid nutritional compound under precisely required speeds and blending times based on the material in the supplement as well as the batch size. The same process can be utilized for preparing topical formulation to achieve enhanced delivery. The amphipathic (hydrophilic and hydrophobic) properties of SK713 SLP allow it to encapsulate nutraceutical ingredients contained in a liquid medium and to serve as an efficient transmembrane delivery vehicle for these nutrients. The SK713 SLP delivery vehicles or spheres as set forth above comprise all natural GRAS (Generally Recognized As Safe) ingredients or pharmaceutically/nutraceutically acceptable ingredients, which are suitable for human consumption.

b. Multilamellar Sphere Components

The SK713 SLP multilamellar spheres contain large quantities of electrolytes and hydroxyl-rich botanicals that contribute bioflavonoids and assist in maintaining healthy pH, proper hydration, and the transport and utilization of vital nutrients. The SK713 SL phospholipid spheres are zwitterions, methyl donors, and potential alkalizing buffer (Bouchard, G., et al., “Theoretical and experimental exploration of the lipophilicity of zwitterionic drugs in the 1,2-dichloroethane/water system,” Pharmaceutical research (2002) 19:1150-1159). Zwitterions carry both positive and negative charges and may lower the energy requirement for transporting molecules thereby enhancing absorption by spreading the nutrient out over a larger surface area.

Zwitterions are soluble in many solvents, e.g. water. The SK713 SL phospholipid spheres have a natural ‘adhesive’ property that enhances the ability of the body to absorb their nutritional contents. Specifically, the embodiment of this invention relates to a novel soy-lecithin-phospholipid-nutrient encapsulation technology, which could achieve rapid onset and improved bioavailability of the nutrients encapsulated within clustoidal multilamellar Soy Lecithin (SK713 SLP) structures.

c. Live Blood Cell Imaging

Live blood cell imaging was performed using an Olympus BX-30 light microscope with a Phase Contrast Condenser to visualize samples. A 150 watt lightbox with fiber optic cable assembly was used to highlight the specimen against a gray field and increase the range of intermediate shades. The lighting produces a high level of cell definition, clearer morphology and can distinguish features of some cell walls. The lens configuration was 10× eyepiece and 100×-oil-immersion objective magnification to achieve approximately 1000 times magnification. Oil immersion achieved finer resolution and brightness.

d. Peripheral Blood Smear Test

Peripheral blood smear was performed by puncturing the finger with a Bayer Single-Let Disposable Lancet 23G 2.25 mm sterile single-use lancing device. A small amount of capillary blood was allowed to exude and collect spontaneously on the fingertip without squeezing the finger. The blood was transferred directly onto a microscope slide without touching the slide with the finger. The slides used were pre-cleaned standard 1 inch by 3 inch with a thickness of 1 mm supplied by Electron Microscopy Sciences. The slide was covered quickly and gently with a cover glass without pressure to protect blood cells from damage. The cover glass was pre-cleaned #1 22 mm×40 mm with 0.13 to 0.17 mm thickness supplied by Electron Microscopy Sciences. The corners of the cover glass were tapped carefully to disperse surface tension and create an even layer for viewing. The slide was then transferred directly to the microscope for viewing. Evaluation of blood properties began in less than 30 seconds after the blood was taken from the finger. Consistent blood extraction and handling procedures were followed to avoid artifacts.

This test is not intended for any diagnostic evaluations as this imaging technology has not been considered appropriate for such applications. Much controversy has arisen over the use of PBS LBCI due to non-adjudicated commercial use, unsubstantiated extrapolations, over-reach and ambiguity of interpretative criteria for diagnostic purposes. The objective of using PBS LBCI in this embodiment was, however, to serve as a time-sensitive marker of biological perturbation and as a visual analytical tool only for the degree of responsiveness of human blood to the delivered bioactive nutrients. As such, the central finding is not the nature of the changes themselves per se, but the extent to which the changes occurred in contrast to the control and baseline groups.

e. Effects on Human Blood

The SK713 SLP encapsulated VMP35 multivitamin formulation was administered transmucosally to thirty-eight (38) human subjects, both males and females, ranging in age from 12 years to 82 years. The blood samples drawn from the testing subjects administered with VMP35 MNC formulation were analyzed and compared to those drawn from subjects in control group, who were administered with commercial available bottled water. The evaluation demonstrates that the SK713 SLP delivery technology exerts rapid positive effects on morphological, hematological, and rheological properties of the blood. Using the subjects administered with water as references, the rapid positive effects of VMP35 MNC formulation include, but not limited to, a breakup of aggregation and splaying out of red blood cell (“RBC”), improved spherical formation of RBC, a progressive reduction (with time) of hypochromicity, improved movement and ability to flow (rheology) of RBCs in the plasma indicating improved hydration, reduced viscosity, reduced surface tension, improvement in protoplasts (a biomarker associated with increased acid burden) from baseline, improved hemoglobin concentration, and a reduction in plasma debris (cleaner blood). Hypochromicity refers to a pale staining red blood cells with broadened central zone of pallor. Such observation most often associates with hypochromic, microcyticanemia, thalassemia, and anemia. The rapid onset of transmucosally administered VMP35 MNC formulation also suggests that the SK713 SLP technology efficiently delivers nutrients into the blood via the sublingual mucosa and may overcome digestive inefficiencies in vivo (Akbarzadeh, A. et al., “Classification, preparation, and applications,” Nanoscale Res. Lett. (2013) 8:102; Akbarzadeh, A. et al., “Synthesis, characterization and in vitro studies of doxorubicin-loaded magnetic nanoparticles grafted to smart copolymers on a 549 lung cancer cell line,” J. Nanobiotechnology (2012) 10: 46; Valizadeh, A. et al., “Quantum dots: Synthesis, bioapplications, and toxicity,” Nanoscale Res. Lett. (2012) 28(7):480).

As set forth above, the presence of embedded free ions in SK713 (prodosome) enhances bio-electrical properties of the liposomal delivery system in an aqueous solution (see Table 2) and in the blood making it superior to conventional phospholipids in terms of its conductive properties and biological compatibility and functionality. Without being bound by any theory, it is believed that the loading of ions and other nutritional ingredients greatly increases the absorption of nutrients and promotes synergistic effectiveness of the simultaneously absorbed nutrients. The molecular structure created in the SK713 liposomal delivery system acts like a biological capacitor that can transport a variety of nutrients simultaneously across the sublingual mucosal membranes in the mouth and/or the wall of the small intestine into the portal circulation.

Application of Prodosome (SK713 SLP) Delivery System in Oral Administration

It is likely that the SK713 SLP spheres provide protection of the encapsulated nutritional contents within the multilamellar structures against the harsh acidic environment in the stomach. This protection enables the nutrients within the spheres to reach the small intestine intact, which promotes greater nutritional synergy in absorption and utilization. The entire SK713 SLP process helps to create a formulation which enables nutrients to disperse over a larger surface area within the small intestine. Initially, the low-sheer tri-blender jet compression technology decreases particle size of larger and more granular or resinous materials. The smaller particle size of a particular nutrient will allow this nutrient to cover a broader surface area once it reaches the small intestine. In addition, encapsulation within the SK713 SLP spheres can decrease particle size even further, especially of fat-soluble vitamins and phytonutrients. As the remaining mass of nutrients that does not absorb through the sublingual mucosa reaches the small intestine, it is likely to be absorbed through diffusion across the epithelial wall of small intestine. The process of decreasing particulate size of these nutrients allows the entire mass of nutrients to disperse over a larger area of the small intestinal wall. This dispersion greatly increases the surface area into which nutrients can be absorbed so that less of the nutritional intake passes into the large intestine for elimination.

Application of Prodosome (SK713 SLP) Delivery System in Transdermal Administration

a. Transdermal Delivery of Human Platelets Encapsulated in Prodosome (SK713 SLP)

As set forth above, in North America, there are pervasive problems of digestive maladies and poor absorption of nutrient. According to the above embodiments, prodosome delivery system is successfully used as transmucosal and oral delivery vehicles. The same prodosome delivery system may be used as a suitable vehicle to delivery beneficial compounds directly to the site of need (e.g. epidermal, dermal and below), or alternatively, to bypass normal digestion and concomitant digestive inefficiency. In another embodiment of this invention, electrolyte-impregnated SK713 SLP delivery system is found suitable for transdermal delivery. Specifically in this embodiment, human platelets were encapsulated in prodosome to form a transdermal delivery system. The SK713 SLP is mixed with the platelet solution in the form of a platelet rich fibrin matrix liquid solution (PRFM). The SK713 SLP is mixed with the PRFM in a sterile vial at a level between 30-60% and agitated by hand for 30 seconds.

In the embodiment, the tissue permeability of prodosome encapsulated human platelets (“PEHPs”) were evaluated using EpiDerm™ in vitro testing system (MatTek Corporation, Ashland, Mass., USA). The EpiDerm™ skin model is a highly differentiated 3D in vitro human skin tissue containing normal human epidermal keratinocytes which are cultured to form a tissue similar to normal epidermis in terms of structure and function. EpiDerm™ has been used since 1993 to evaluate the dermal irritancy of products applied to the skin. Companies such as Procter and Gamble, Johnson & Johnson, Unilever, Clairol, and L'Oreal have published or presented their work on utilization of the EpiDerm™ skin tissue to predict dermal irritation. In addition, EpiDerm™ has also been used extensively in assessing the performance of transdermal delivery vehicles, including skin corrosion, skin hydration, dermal drug delivery, phototoxicity, and dermal genotoxicity.

In this embodiment, PEHPs were further stained with monoclonal antibody, which binds to platelets biomarkers, CD42b and CD62p antigens. The location of the stained platelets when permeating through EpiDerm™ artificial skin tissues can be tracked using a confocal microscopic imaging technique. In addition to staining CD42 antigens, other biomarkers of EpiDerm™ tissues, e.g. fibrin, IL-6, IL-8, IL-1β, MCP-5, and VEGF, were analyzed to evaluate the effects of PEHPs exposure on the artificial skin tissues.

Fibrin is an insoluble, non-globular protein involved in clotting of blood. Polymerized fibrin becomes entangled with platelets to form blood clots.

IL-6, IL-8, IL-1β are important inflammatory cytokine proteins, which play important roles in wound healing.

MCP-5 is a novel and potent monocyte active chemokine that is involved in allergic inflammation and the host response to pathogens.

VEGF is a vascular endothelial growth factor, which stimulates wound healing.

Epiderm™ tissues can be evaluated using histological analysis. Histological analysis takes photomicrographs of Hematoxylin and Eosin (“H&E”) stained cross-sectional tissue at certain time points to evaluate structural disruption and abnormal tissue staining.

As described above, confocal microscopic imaging analysis, cytokine analysis, and histological analysis were performed at given time points for this embodiment. The analytical results surprisingly revealed that human platelets encapsulated in prodosome delivery vehicle migrated from the stratum corneum (apical cells) to the basal cell side of Epiderm™ tissue model. In the process of PEHPs migration, the release of fibrin and IL-6, the two cytokine biomarkers, from Epiderm™ tissues significantly increased in comparison to the control groups (not exposed to PEHPs) indicating onset of wound healing. Over the same period of time, histological results showed that the exposure to PEHPs did not induce structural damages or significant changes in Epiderm™ tissue morphology.

b. Transdermal Delivery of Lidocaine Encapsulated in Prodosome

In another embodiment of this invention, lidocaine was encapsulated in prodosome to form a transdermal delivery system. The method of preparing this embodiment was the same as those set forth above.

The permeation of prodosome encapsulate lidocaine (“PEL”) was evaluated using Epiderm™ model skin tissues. The permeation of PEL across Epiderm™ model was analyzed using LC/MS/MS technique. The structural integrity of Epiderm™ model tissues in the permeation studies of PEL was evaluated using histological analysis.

Permeation results showed a four-fold increase in permeation at 24 hours in comparison to the 2 hours and 4 hours exposure time points. The four-fold increase in lidocaine permeation at 24 hours exposure time corresponded to an approximate 75% absorption of the drug through the epidermal layer of the reconstructed tissue model. Over the same period of time, histological results showed that the exposure to PEL did not induce structural damages or significant changes in Epiderm™ tissue's morphology.

The two embodiments relating to transdermal prodosome delivery system as set forth above demonstrate that electrolyte-impregnated SK713 SLP is a suitable vehicle for transdermal delivery of both small molecules, e.g. lidocaine, and relative large particles, e.g. human platelets. In addition, according to the histological studies of the above two embodiments, prodosome based transdermal delivery technology is safe to the skin and does not significantly alter the skin structure.

c. Transdermal Delivery of Other Active Ingredients Encapsulated in Prodosome.

As set forth in the above two embodiments, electrolyte-impregnated prodosome delivery system was demonstrated to be a suitable vehicle for transdermal delivery of both small molecules such as lidocaine and large particles such as platelets. The above two embodiments are not intended to be limiting and the suitable active ingredients that can be transdermally delivered using prodosome vehicle include, but are not limited to, NSAIDS (e.g. ibuprofen), antibiotics, and insulin, anesthetic agents, chemotherapeutic drugs, acne medications, vaccines, blood thinners, etc.

In addition, the embodiment of prodosome encapsulated lidocaine as set forth above is also suitable for the delivery of lidocaine to a subject's ears, wherein the subject is a human or an animal. For example, encapsulated lidocaine may be delivered to the ear canal, or through the ear canal to inner ear tissues by medical professionals.

Overall, all the embodiments set forth above can be prepared, shipped, and stored as liquid suspensions, which are ready to use by a subject in need. However, the same embodiments can be also prepared in solid dosage forms, e.g. through freeze drying/lyophilization. The solid dosage forms of this invention can be reconstituted by a medical practitioner or a subject in need before administration, wherein the subject is a human or an animal.

The multi-lamellar prodosome compositions and methods described above, the effect of prodosome encapsulated VMP35 MNC on human blood, prodosome encapsulated human platelets for transdermal delivery, and prodosome encapsulated lidocaine for transdermal delivery may be further understood in connection with the following Examples. In addition, the following non-limiting examples are provided to illustrate the invention.

### Example 1

### Method of Producing Clustoidal Multilamellar Soy Lecithin Phospholipid (SLP)

Step 1. Generally, a nutritional, nutraceutical, or pharmaceutical active ingredient substrate is processed through an advanced wet milling/particle compression process to facilitate a type of mechanical predigestion of substrate that enables more of the substrate to be encapsulated in the phospholipid spheres. Thoroughly wet milling the substrate significantly increases surface area of the substrate and enables a higher concentration and wider range of substrate ingredients to be homogenized and encapsulated in the Prodosome process.

The following steps are done in relatively small batches (approx. 5 gallon containers) to achieve an optimal speed ensuring the most complete and thorough homogenization of constituents. Following each step below, blending should be performed in small circular motions in the opposite direction of the rotation (counter-rotation) of the blender blade to increase the torsion to effect the interaction of ions with phospholipids over a greater fluid surface area and produce an energetically enhanced homogenous mixture. Generally, start with an amount of water between 40-80% of total final volume. Heat water to a temperature between 90 degrees F. to 140 degrees F.

Step 2. In a 5 gallon stainless steel drum of water, solar evaporated mineral/trace mineral liquid concentrate between 1 to 120 g/kg of water was mixed in at a level ranging from 0.1% to 12.0%. This mixture was blended for a time between 1-5 minutes at a speed between 3,000-25,000 RPM in a high-RPM spinning vortex of water between 300 to 800 g/kg of total mixture to completely and uniformly disperse ions into what is now ‘structured water.’ (Trace mineral liquid concentrate is available from Trace Minerals Research, Ogden, Utah; see also FIG. 16.)

Step 3. High-grade lecithin containing >85% Phosphatidylcholine (PC) 2 to 200 g/kg of total mixture was added at 2 to 20% and thoroughly mixed into the ion-rich water, blended between 1-5 minutes at a speed of 3,000-25,000 RPM, depending on substrate viscosity. Then, a small amount of ethyl alcohol was added (NLT 150 proof) at 50 to 450 g/kg of total mixture and blending continued between 1-5 minutes at a speed of 3,000-25,000 RPM depending on substrate viscosity. The mixture is then allowed to cool. As a result, the phospholipid structures are completely impregnated and saturated with free ions, achieving a completely homogeneous mixture of electrolytically ‘charged’ SK713 SLP material.

Variants of the procedure include: Adding between 2-20% amounts of phosphatidyl choline with a PC content of no less than 70%. Adding between 5-45% USP Alcohol, at a level no less than 150 proof. The mixing procedure can include ultrasonic mixing.

Step 4. This mixture is then added to the nutritional, nutraceutical, or pharmaceutical active ingredient substrate of Step 1 in a blender and blended thoroughly to facilitate complete encapsulation of the substrate. A level of 0.5% to 10% of the present invention can be used in ‘prodosoming’ finished products depending on the composition and state (aqueous or dry) of the substrate being encapsulated.

The process may be varied slightly, within a narrow parameter, as to the degree of phosphatidyl choline (PC) content, depending on the end usage required. Limited variance of PC content of finished Prodosome may alter viscosity of liposomal material without creating any loss of advantage. Differing viscosity Prodosomes may be required depending on active ingredient intended for encapsulation, such as material more or less soluble, or materials containing higher level of lipids. Trace mineral concentrate amounts can also be varied to some extent, depending on the substrate and benefit endpoints.

This mixing process evidently catalyzes association between electrolytes and other molecules within the total substrate (i.e. methyl and phosphoryl groups); certain B vitamins with methyl and/or phosphoryl ligands; also facilitating the permeation of substrate material into the phospholipid intermolecular spaces of the Prodosomes.

This process enables comprehensive and uniform encapsulation of nutritional and/or pharmaceutical ingredients in the SK713 SLP phospholipid prodosome capsules facilitating superior absorption of nutritionally and pharmacologically active therapeutic substances that provide benefits following absorption of the energetically enhanced electrolyte-impregnated phospholipids.

The present disclosure comprises specific materials with exacting levels of each, blended with distinct sequence and timing. The SK713 sphere is unique in many aspects, as follows.

A. Higher levels of PC-rich lecithin help to ensure stability and more comprehensive encapsulation.

B. Mixing of total compound in smaller containers, thereby allowing more thorough and uniform blending. This is as opposed to typical mixing on larger scales which hampers proper fluidization.

C. Part of the total methodology of this invention requires pre-treatment of nutrients to be encapsulated. This can include but is not limited to wet milling, or partial dissolution using low or high shear wet milling (depending on substrate to be milled) to make active ingredients uniformly smaller and more accepting of the invention's encapsulation. This method also protects the integrity of the active compound being treated.

D. Other important reasons for mineralizing the water are decreasing zeta potential and improving stability. Typical water used in pharmaceutical/nutraceutical manufacturing is distilled through de-ionization or reverse osmosis. This form of water, while pure, typically has aggressive receptor properties vs. aggressive donor properties. As a ‘receptor’ it can become acidified by complexing with CO₂ (for example) as well. Empty, aggressive reception, and/or acidified water can disrupt surrounding mediums, including aqueous mediums containing nutrients. By aggressively mixing the water in a consistent vertical motion, the water becomes more structured. This motion also stabilizes the water portion of the liposomal sphere with added electrolytes, which causes the water to become more biocompatible, stable and less disruptive to the nutrients contained therein. Therefore the entire final prodosome structure is more stable.

E. The invention starts with pharmaceutical grade water to ensure purity, and then adds a precise pre-measured amount of mineral electrolytes at the appropriate time to ‘mineralize’ the water as just indicated above. This process ensures uniformity of mineral levels and distribution during each production process and also ensures a finished compound that has more of the biocompatible properties of body fluids and more readily promotes competent cell metabolism. Also, unlike relying on mineral water from a natural source, which can have impurities, varying potencies of minerals, and a complete absence of one or more mineral compounds, the process of the present invention ensures that the mineral electrolytes are supplied in uniform, ample, and comprehensive amounts. To this point, a 30-50 gallon batch of finished product was allowed to sit for 7 hours and experienced an exothermic reaction in which the temperature of the batched product rose to 98.6 degrees Fahrenheit, i.e. the temperature of body fluids, and then stopped. The present invention is creating a specific resonance that is completely biocompatible with body fluids.

F. The invention's inclusion of trace minerals contributes to intracellular pH regulation and homeostasis and pH stability in the liposomal sphere contained within the product prodosome, especially important because enveloped nutrients (e.g., Vitamin C) may disrupt pH balance. By avoiding this circumstance, additional stability is provided for the liposomal sphere contained within the product prodosome. Furthermore, the ability of the SK713 liposomal sphere contained within the product prodosome, infused and saturated with our special mineral rich electrolyte material, is that the sphere can impart, through the action of mineral buffering, a pH balancing effect within the bloodstream concurrently with the release of the contained nutrients. It should not be inferred that the pH of the SK713 or its substrate impose any buffering effects because of their pH properties. Rather, the SK713 and the ionic constituents contribute buffering potential as needed for the body's homeostatic requirements. This phenomenon can improve cellular uptake and utilization of available nutrients.

Other known liposomal technologies are plagued with instability; gradual and continual degradation of liposomal capsules; and substrate ‘leakage’ out of degrading and delineating liposomes ultimately results in a reduction and eventual loss of liposomal encapsulating benefits. Evidence of this degradation are visible in product containers as solid residues continue to amass, precipitate and accumulate on the bottom of the containers. In contrast, thoroughly and completely “Prodosomed” product remains completely and evenly dispersed and homogenized throughout the blended mixture. The SK713 process helps to ensure that capsule stability, homogeneity, and therefore stronger and more sustained benefits occur from products treated with prodosomes in the embodiments of the invention.

Surface Tension Measurement

Other beneficial properties are evidenced by the Surface Tension testing done on standard liposomes vs. Prodosomes as prepared in Example 1. Testing was performed by NSL Analytical. Two liquid samples were submitted for Contact Angle measurement on a glass slide surface. The test outlined was performed on both samples. The measurements were recorded at five seconds intervals due to the small area of contact. Once the drop (10 μl) was in contact with the surface the first measurement was recorded and the second measurement was recorded after approximately five seconds and the same for the third, fourth and fifth. Sample #1 (standard liposome) demonstrated an average Contact Angle of 39. Sample #2 (Prodosome) demonstrated an average Contact Angle of 47.7. The inclusion and specific mixing process of the trace minerals into the Prodosomes increased the average level of surface tension by 22.3%. The increased surface tension has a direct and significant impact on liposomal integrity and can be attributed to the SK713 Process which as previously discussed increases Zeta Potential thereby reducing agglomeration and increasing the dispersion and subsequent stability of the solution. A higher Zeta Potential leads to a stronger level of electrostatic repulsion within the solution and subsequent stronger liposomal shell(s) in the clustoidal multi-lamellar SLP prodosome structure of Example 1.

Advantages produced by this process include increased stability of the liposomal transport sphere contained within the product prodosome while simultaneously not adding to the cost or burden of producing the material. It also affords an increased opportunity to enhance cellular uptake of nutrients, both by balancing extracellular and intracellular pH and by bolstering extra- and intracellular fluid exchange. These actions occur concurrently with the delivery of nutrients, which creates additional synergies to benefit health. A replenishment of electrolytes is vital to maintaining a balanced osmotic gradient within plasma to ensure optimal oxygenation, correct hydration via maintaining optimum pH. It is this correct hydration and pH that affects all other usage of nutrients delivered by the liposomal sphere contained within the product prodosome.

The process as described herein is focused on a new paradigm of altering the functionality of the liposome giving it a dual purpose. With the SK713 Prodosome, the liposome now acts as both a delivery vehicle and a functional enhancer of the receptor or target of the delivered materials.

Other advantages also include low cost of production; ease of transport for usage on site; no additional or unusual equipment needed for usage; able to be stored at room temperature; better stability of SK713 material and better stability of liposomal material containing enveloped nutrients within the product prodosome; process uses pre-preparation of active ingredients to be Prodosomed in order to ensure better and more thorough encapsulation; and the creation of electrically charged, energy-enhanced phospholipids of the Prodosome which acts as a transport vehicle while also actively influencing cellular integrity for enhanced utilization of nutrients.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

### Example 2

### Effect of SK713 SLP Encapsulated VMP35 Multivitamin Formulations on Human Blood

Experimental Design

SK713 SLP encapsulated VMP35 MNC formulation was prepared using the method described in Example 1. This example relates to a controlled cross-over study to evaluate the effects of transmucosal administration of SK713 SLP encapsulated VMP35 MNC (active) as opposed to baseline and commercially available bottled water (control). Thirty-eight (38) subjects were recruited from random interviews. There were ten (10) males and twenty-eight (28) females ranging in age from twelve (12) years to eighty-two (82) years with an average age for males of forty-nine (49) years and for females of forty-six point eight (46.8) years as seen in Table 5. Subjects were assigned randomly into one of three groups (baseline, control, and active) and underwent peripheral blood smear (PBS) live blood cell imaging (LBCI) as shown in Table 6. The baseline blood samples were drawn from all the subjects prior to transmucosal administration of VMP35 MNC formulation or transmucosal administration of water to the same subjects. Changes in peripheral blood smear (PBS) were examined using Live Blood Cell Imaging and Phase Contrast Microscopy. (Popescu, G., et al., “Imaging red blood cell dynamics by quantitative phase microscopy,” Blood cells, molecules & diseases (2008) 41:10-16).

TABLE 5
Randomly Selected Subjects Participating in Live Blood Cell Imaging

| Participant | Age | Gender | Ethnicity | Self-Reported Health Issues |
|---|---|---|---|---|
| 10 | 37 | Female | Guyanese | None |
| 11 | 45 | Male | Caucasian | High Blood Pressure (BP) |
| 12 | 16 | Female | Caucasian | None |
| 13 | 13 | Female | Caucasian | None |
| 14 | 37 | Female | Caucasian | Allergies |
| 15 | 43 | Female | Caucasian | Poor Digestion |
| 17 | 70 | Female | Italian | Osteoporosis, Arthritis |
| 18 | 24 | Male | Lebanese | None |
| 19 | 22 | Female | Caucasian | None |
| 20 | 22 | Female | Caucasian | None |
| 21 | 61 | Male | Caucasian | None |
| 22 | 51 | Female | Caucasian | None |
| 23 | 37 | Male | Caucasian | None |
| 24 | 62 | Female | Caucasian | Skin Condition |
| 25 | 54 | Female | Caucasian | None |
| 26 | 63 | Female | Caucasian | Diabetes |
| 27 | 58 | Female | Caucasian | None |
| 28 | 43 | Male | Caucasian | Digestion Problems |
| 29 | 49 | Female | Caucasian | None |
| 30 | 51 | Female | Caucasian | None |
| 31 | 24 | Female | Caucasian | Attention Deficit Disorder. |
| 32 | 61 | Female | Caucasian | Thyroid, Severe Pain |
| 33 | 56 | Female | Caucasian | None |
| 34 | 60 | Female | Caucasian | None |
| 35 | 58 | Female | Caucasian | Depression, Thyroid, |
| | | | | Hormone |
| 36 | 79 | Female | Caucasian | High BP, Diabetes, Heart |
| 37 | 35 | Female | Caucasian | None |
| 38 | 40 | Female | Caucasian | None |
| 39 | 57 | Female | Caucasian | None |
| 40 | 12 | Female | Caucasian | Skin Condition |
| 44 | 44 | Male | Trinidadian | None |
| 45 | 50 | Male | Italian | None |
| 46 | 50 | Female | Caucasian | Toxic Exposure |
| 47 | 71 | Male | Caucasian | Severe Periodontal Disease |
| 48 | 74 | Female | Italian | High Blood Pressure |
| 49 | 82 | Male | Italian | Bladder Cancer, Cl1 |
| 50 | 33 | Male | Caucasian | Herpes |
| 51 | 56 | Female | Italian | None |

TABLE 6
Group of Subjects and Blood Test Time

| | | 5 minutes after | 5 minutes after | 30 minutes after |
|---|---|---|---|---|
| Groups (n) | Baseline | water | VMP35 MNC | VMP35 MNC |
| Group 1 (n = 8) | Group 1 | Group 1 (control) | Group 1 (active) | |
| Group 2 (n = 23) | Group 2 | | Group 2 (active) | |
| Group 3 (n = 7) | Group 3 | | Group 3 (active) | Group 3 (active) |
| Total tests | 38 | 8 | 38 | 7 |

After taking baseline blood samples, PBS Group 1 (n=8) consumed 30 mL water with a follow-up PBS taken at 5 minutes. The moment of administration of water or VMP35 MNC formulation to a subject is used as time zero. Both active groups Group 2 (n=26) and Group 3 (n=7) consumed 30 mL of VMP35 MNC with a follow-up PBS taken at 5 minutes. Thereafter, Group 3 had an additional PBS taken at 30 minutes. Group 1 then consumed 30 mL of VMP35 MNC and had a PBS at 5 minutes after intake. The dosing regimen and sampling schedule are summarized below.

Group 1: Water Control group consisting of 8 individuals (3 blood samples each):
a. Baseline blood test prior to the intake of water
b. 2nd blood test at 5 minutes after the intake of water
c. 3rd blood test at 5 minutes after the administration of VMP35 MNC

Group 2: Active Group consisting of 23 individuals (2 blood samples each):
a. Baseline blood test prior to the administration of VMP35 MNC
b. 2nd blood test at 5 minutes after the administration of VMP35 MNC

Group 3: Active Group consisting of 7 individuals (3 blood samples each)
a. Baseline blood test prior to the administration of VMP35 MNC
b. 2nd blood test at 5 minutes after the administration of VMP35 MNC
c. 3rd blood Test at 30 minutes after the administration of VMP35 MNC

Results

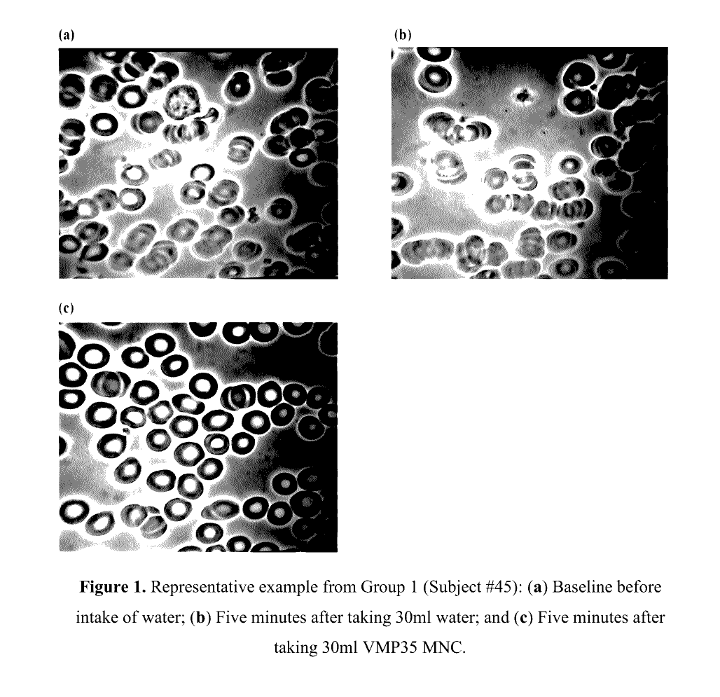

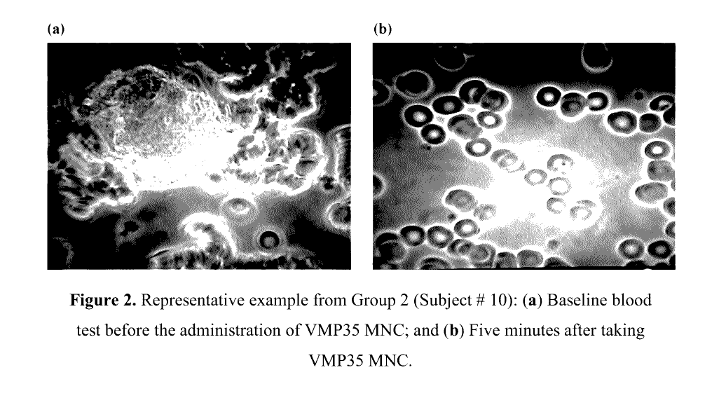

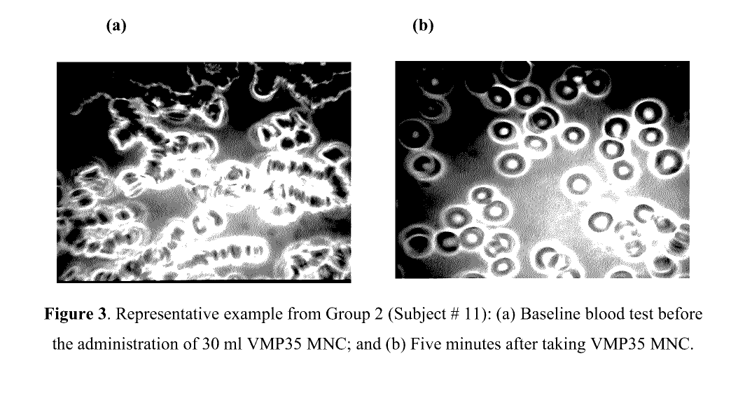

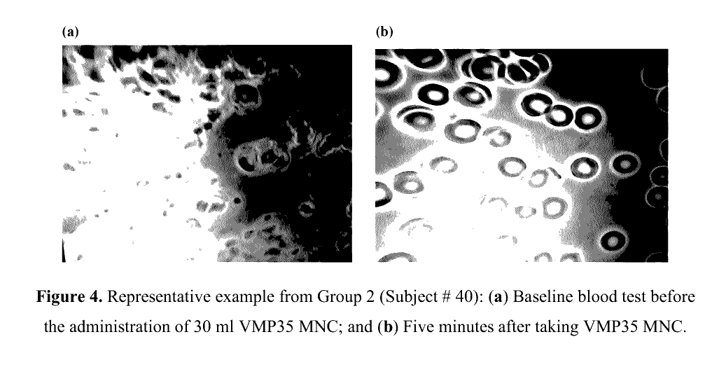

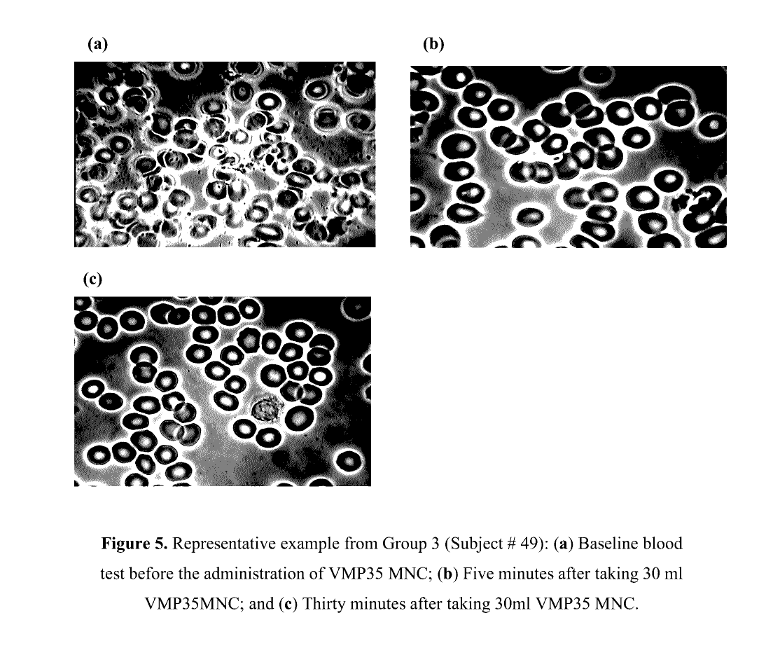

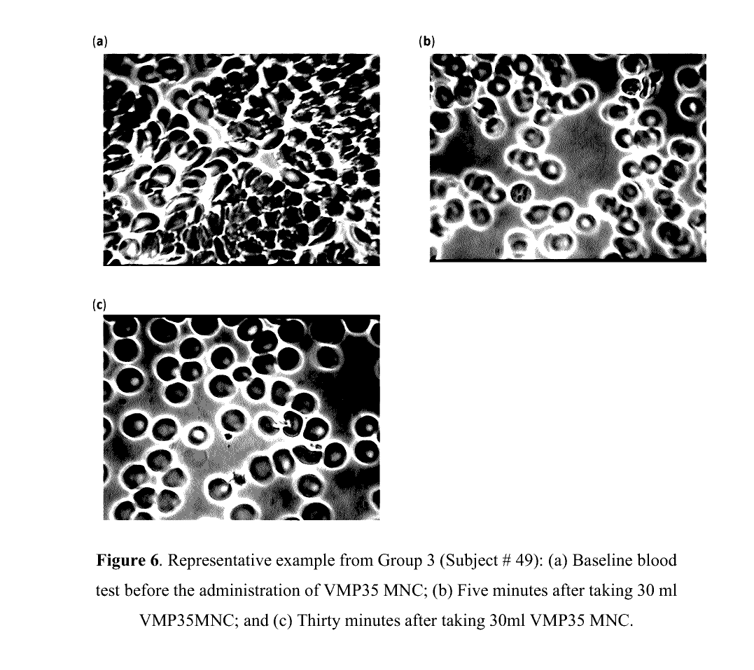

A non-blinded comparison was done between the baseline and subsequent PBS samples. Pictures were taken for blood samples during each phase of the study. For each group, changes in morphological, hematological and rheological characteristics were recorded. Representative results are depicted in FIGS. 1-6. Specifically, FIGS. 1(a) and (b) indicate that no changes were observed between the baseline and the 5-minute samples in the control group (Group 1). Substantial differences were observed between the baseline and 5-minute samples in the active Groups 1 and 2. (See FIGS. 1(b) and (c), FIGS. 2 (a) and (b), FIGS. 3 (a) and (b), and FIGS. 4 (a) and (b)). Substantial differences were observed among the baseline, 5-minute, and 30-minute in the active Group 3. (See FIGS. 5 (a), (b) and (c), and FIGS. 6 (a), (b) and (c)). Improvements in the splayed arrangement, size, form, density and distribution of RBCs following intake of the VMP35 MNC can be clearly identified in these figures and are indicative of improved morphological, hematological, and rheological properties.

Baseline and Control

Images of red blood cells (RBCs) obtained from baseline and the 5-minute samples in the control group clearly showed aggregation and immobility—a sludge effect, malformation and damage, and extensive hypochromic state (i.e. an oversized ‘donut hole’ evidencing reduced hemoglobin). In the images of baseline samples, protoplasts (a biomarker associated with increased acid burden), extensive ‘debris’ in the plasma, and ‘dwarfed’ white blood cells (WBCs) were also observed.

RBC Improvements 5 Minutes after the Administration of VMP35 MNC

RBC improvements 5 minutes after the administration of VMP35 MNC (shown in FIGS. 1-6) included a breakup of aggregation and splaying out of RBCs on the slide, improvement in spherical formation of RBC, and a progressive reduction (with time) of hypochromicity. Other positive effect of transmucosal VMP35 MNC included improved movement and ability to flow (rheology) of RBCs in the plasma, evidencing improved hydration, reduced viscosity, and reduced surface tension.

RBC Improvements 30 Minutes after the Administration of VMP35 MNC

LBCI results of Group 3 at 5 minutes and 30 minutes post intake of VMP35 MNC (shown in FIGS. 5 and 6) were similar to each other, both of which showed improved hemoglobin concentration, a reduction in plasma debris (cleaner plasma), and reduced quantity of protoplasts.

Overall, RBC and blood rheology improvements observed in this example demonstrate that SK713 encapsulated VMP35 MNC formulation can be absorbed and delivered to the blood within 5 minutes through sublingual transmucosal administration. The central finding of this example is the fact that the improvements occurred within 5 minutes after the administration of VMP35 MNC formulation and were sustained for at least 30 min. Conversely, no such changes were found when the equivalent volume of water was ingested by the control group, which adds credibility to the baseline findings and demonstrates reproducibility in the absence of active intervention. On the other hand, the prompt, sustained and progressive findings in Group 2 at 5 minutes and Group 3 at 5 and 30 minutes offer support that the observations were also valid metrics to observe the bioactive effects. This conclusion is further strengthened by the appearance of the same results in Group 1 during the active cross-over phase (switching to VMP35 MNC formulation).

This example demonstrates that the SK713 SLP delivery technology exerts rapid positive effects on morphological, hematological, and rheological properties of the blood. This rapid response also suggests that the SK713 SLP technology efficiently delivers nutrients into the blood via the sublingual mucosa, in less than 5 minutes from intake and may overcome digestive inefficiencies in vivo.

### Example 3

### Permeation of Prodosome Encapsulated Human Platelets (“PEHPs”) in Epiderm™ Skin Model

Materials

The EpiDerm™ (EPI-200X) human tissue produced by MatTek Corporation was used. The EPI-200× tissue lot used for this study met QC acceptance criteria and the positive/negative controls.

Experimental Procedures

EpiDerm™ tissues were pre-incubated for 1 hour at 37° C.±1° C. and 5%±1% CO₂ in 6-well plates containing 0.9 ml of assay medium. Tissues were removed from the incubator and re-fed with pre-warmed assay media. Human platelets (500,000-750,000 per 100 μl) were mixed with prodosome delivery vehicle in a 1:1 (v:v) ratio to form PEHPs. The average size of human platelets used in the experiment was about two microns. Subsequently, 100 μl PEHPs was applied topically to EpiDerm™ (EPI-200X) tissue. For each time point, two PEHPs samples were taken (N=2). For each time point 2 untreated tissues were also used to serve as untreated controls. Prodosome vehicles without loading human platelets were also applied to Epiderm™ tissues and used as vehicle control. After 2, 4, and 24 hours of exposure time points, culture supernatants were collected and stored at −70° C. until analysis. Two samples were taken at each time point (N=2). Tissues were rinsed with PBS, fixed in formalin for 24 hours, paraffin-embedded, cryosectioned, and used for H & E staining and immunohistochemistry (IHC) using standard methods.

Confocal Imaging

To evaluate the localization of platelets, cryosections were prepared from untreated, vehicle control, and platelet treated samples. The cryosectioned tissues were subsequently formalin fixed and stained for platelet markers (CD42b and CD62p) using standard methods.

Cytokine Analysis

At time of 24 hours post exposure, culture supernatants were collected from the platelet treated and untreated EpiDerm™ tissues. Releases of biomarkers, e.g. fibrin, IL-6, IL-8, IL-1β, MCP-5, and VEGF, from culture supernatants were analyzed using ELISA assays, which are well-known in the art.

Histological Analysis

At time of 2, 4, and 24 hours post exposure, tissues were gently washed in PBS to remove any remaining test material from the surface of the tissues, formalin fixed, paraffin embedded, cross-sectioned, and hematoxylin and eosin (H&E) stained. A slide per tissue sample was stained with H&E.

Results

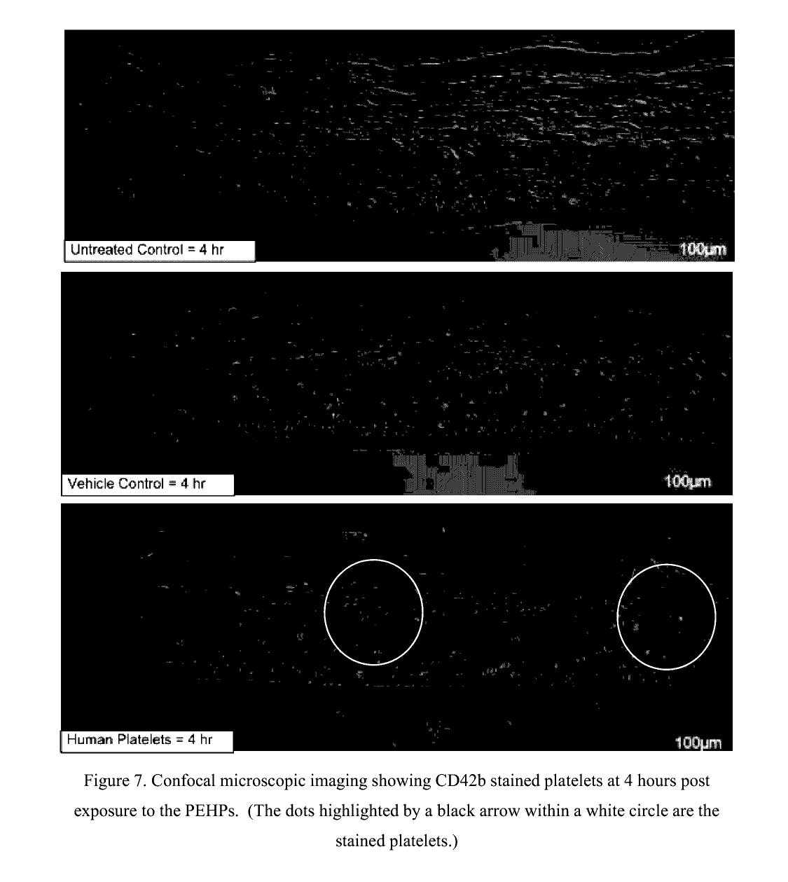

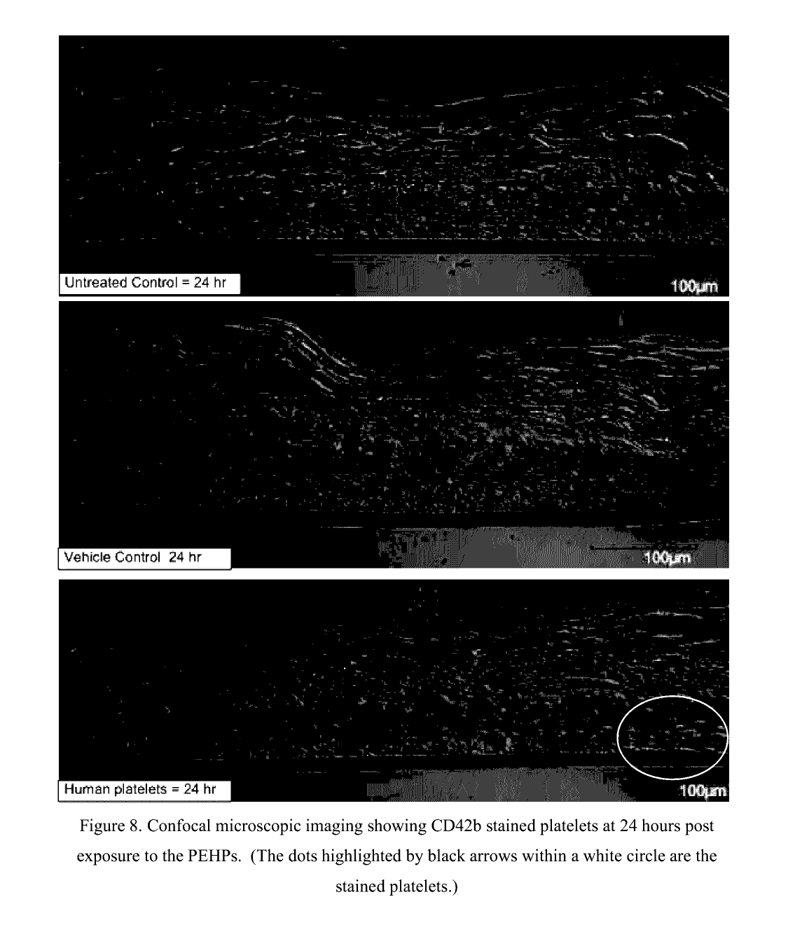

Confocal imaging was performed using monoclonal antibody to bind platelet glycoprotein Ib alpha chain (GPIb alpha), also known as CD42b alpha. The results showed few CD42b positive staining just below the stratum corneum at time of 4 hours (FIG. 7). At time of 24 hours, weakly CD42b stained platelets were observed at the basal cell side of the tissue model (FIG. 8). All tissues (controls and platelet exposed) were negative for the platelet activation marker, CD62p (data not shown). CD62p is a 140 kD type I transmembrane glycoprotein, also known as P-selectin, platelet activation-dependent granule membrane protein (PADGEM). It is expressed on activated platelets, megakaryocytes, and endothelial cells.

Cytokines analysis following PHEPs exposure revealed:

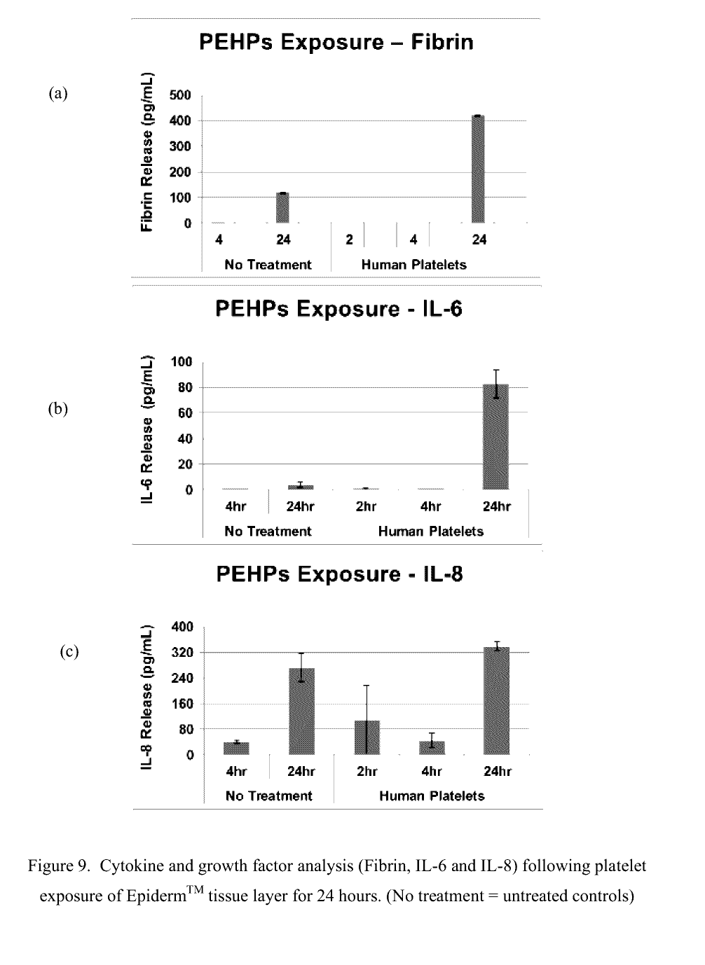

(1) No fibrin release was observed at 4 hours. At time of 24 hours, the PEHPs exposed tissues showed a four-fold increase in fibrin release compared to untreated controls; (See FIG. 9(a)).

(2) No IL-6 release was observed at 4 hours. At time of 24 hours, the PEHPs exposed tissues showed a significant increase in IL-6 release compared to untreated controls; (See FIG. 9(b)).

(3) No significant difference was observed in IL-8 release between the PEHPs exposed and unexposed control issues at all time points tested; (See FIG. 9(c)).

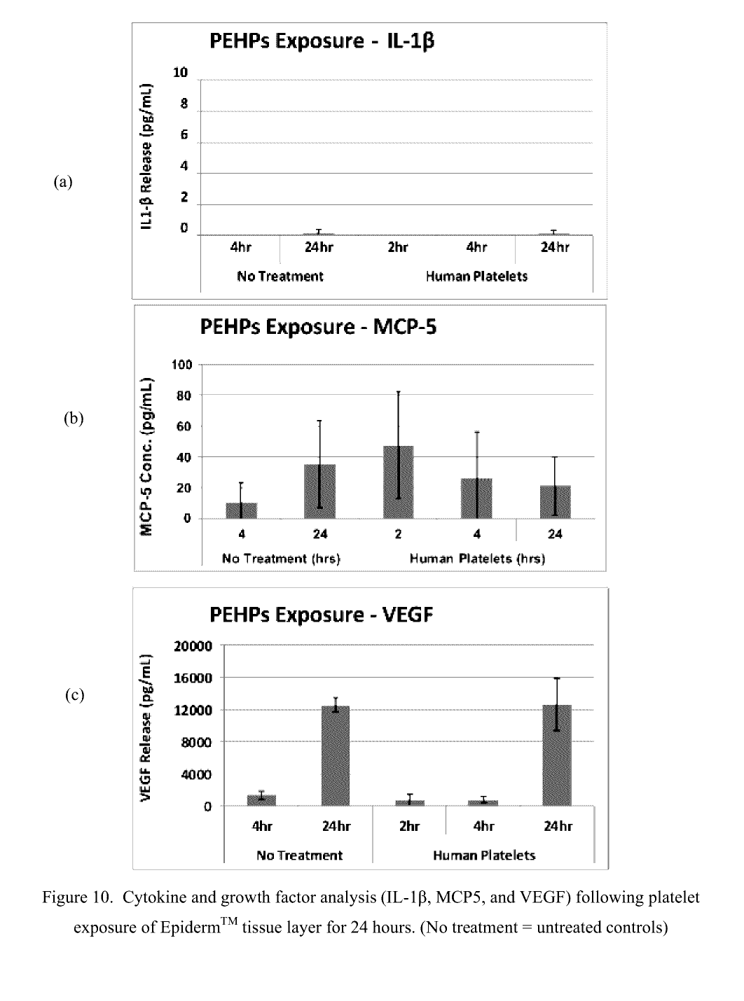

(4) No significant difference was observed in IL-10 release between the PEHPs exposed and untreated control issues at all time points tested; (See FIG. 10(a)).

(5) No significant difference was observed in MCP-5 release between the PEHPs exposed and untreated control issues at all time points tested; (See FIG. 10(b)).

(6) No significant difference was observed in VEGF release between the PEHPs exposed and untreated control issues at all time points tested. Since VEGF is both expressed and secreted by epidermal keratinocytes, the observed values at time of 24 hours are considered background levels. (See FIG. 10(c)).

Since IL-6 is one of the important inflammatory cytokines implicated in wound healing, the relative increase in IL-6 level coupled with fibrin release following topical application of prodosome encapsulated human platelets suggests a potential use of the prodosome technology in wound closure and repair in the skin microenvironment.

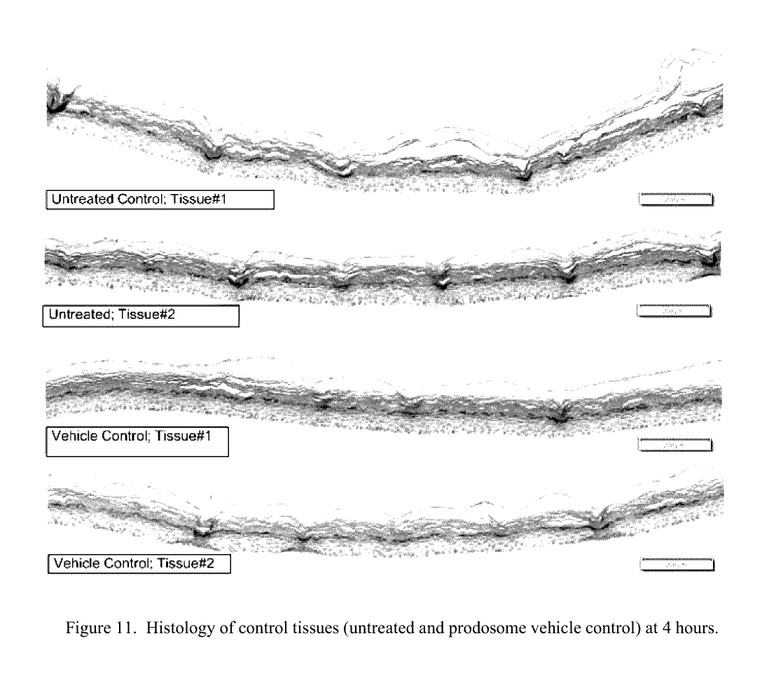

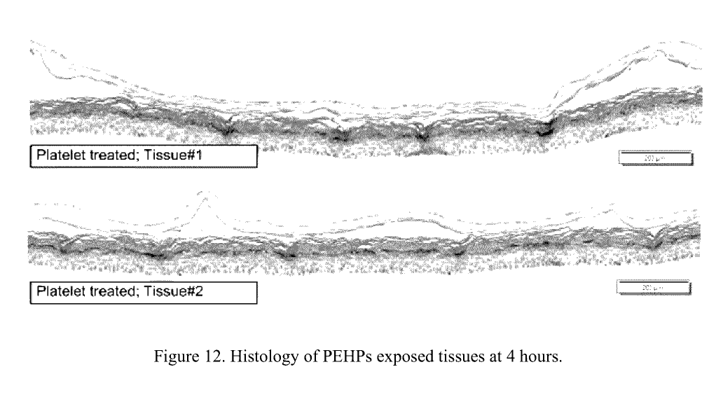

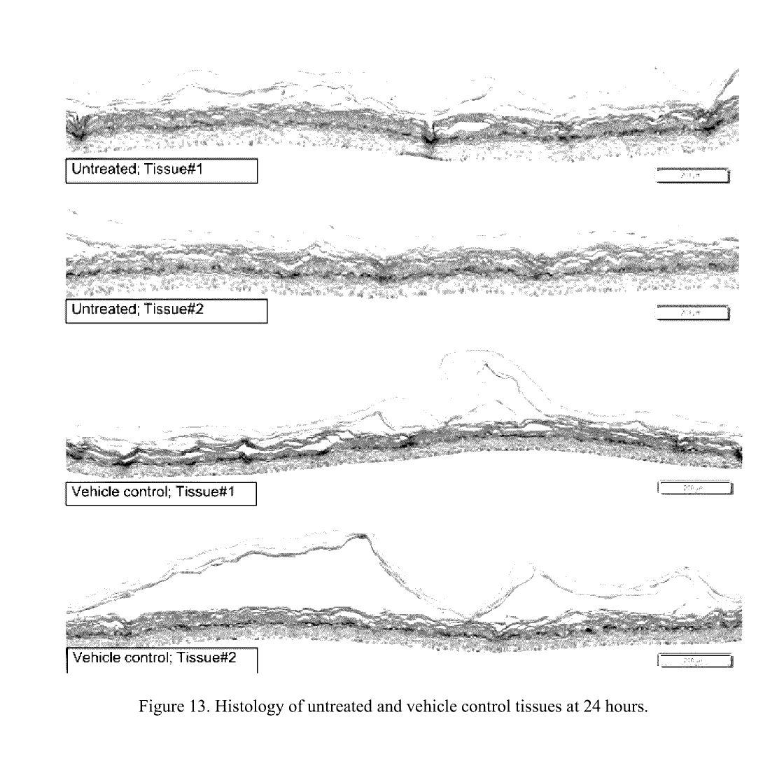

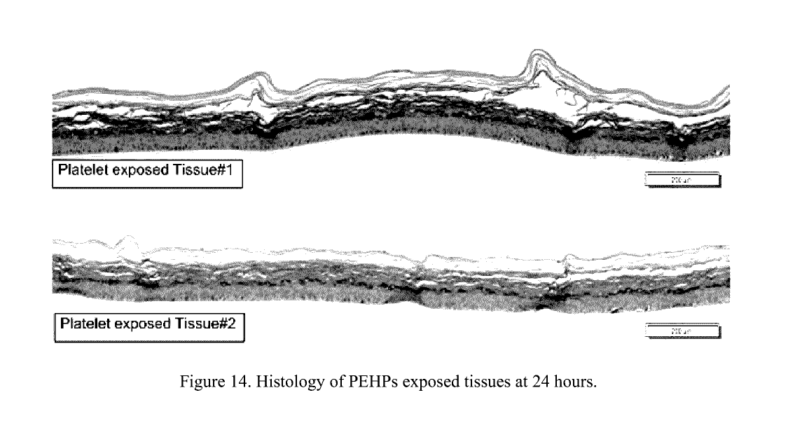

Photomicrographs of H&E stained histological cross-sections of the EpiDerm™ tissues following a 4-hour and a 24-hour exposure to PEHPs and controls are shown in FIGS. 11-14. The cross-sections of EpiDerm™ tissues were evaluated for disruption of the apical tissue layers, structural disruption, and abnormal tissue staining. Among untreated control EpiDerm™ tissues, there were no apparent structural damage or significant changes in tissue morphology at 4 hours or 24 hours. (See FIGS. 11 and 13). Among vehicle control EpiDerm™ tissues, no apparent structural damage or significant changes in tissue morphology were observed at any time points. (See FIGS. 11 and 13). Among PEHPs exposed EpiDerm™ tissues, no apparent structural damage or significant changes in tissue morphology were observed at any time points. (See FIGS. 12 and 14).

### Example 4

### Permeation of Prodosome Encapsulated Lidocaine

### Experimental Procedures

Normal 1% lidocaine hydrochloride solution (1000 μg lidocaine hydrochloride/100 μl solution or 810 μg lidocaine/100 μl solution) was mixed in a 1:1 (v/v) ratio with the prodosome delivery vehicle. 100 μl of the resulting prodosome encapsulated lidocaine (405 μg of lidocaine in 100 μl) was applied topically to the Epiderm™ tissues. Culture supernatants collected at 2, 4, and 24 hours after PEL exposure were analyzed for lidocaine permeation. The concentrations of lidocaine in culture supernatants were determined using LC/MS/MS (Agilent 6410 mass spectrometer).

LC/MS/MS Analysis

Samples were analyzed by LC/MS/MS using an Agilent 6410 mass spectrometer coupled with an Agilent 1200 HPLC and a CTC PAL chilled auto sampler, all of which were controlled by MassHunter software. After separation on an HPLC column (Agilent Zorbax SB-C18 2.1×30 mm 3.5 u, 120 A) using an acetonitrile-water gradient system, peaks were analyzed by mass spectrometry (MS) using ESI ionization in MRM mode. Cell culture media samples were processed with three volumes of methanol containing internal standard (propranolol). Samples were then centrifuged to remove precipitated protein or salt, and the supernatant was analyzed by LC-MS/MS. Lidocaine concentrations in cell culture media samples were quantified using a calibration curve prepared in cell culture media.

Results

TABLE 7
Concentration of Lidocaine in Cell Culture Media

| | Conc. |
|---|---|
| Group | Lidocaine (μg/mL) |
| 2 hr_1 | 67.1* |
| 2 hr_2 | 13.9 |
| 4 hr_1 | 60.0* |
| 4 hr_2 | 19.4 |
| 24 hr_1 | 311** |
| 24 hr_2 | 303** |

*based on 10-fold dilution;
**based on 50-fold dilution

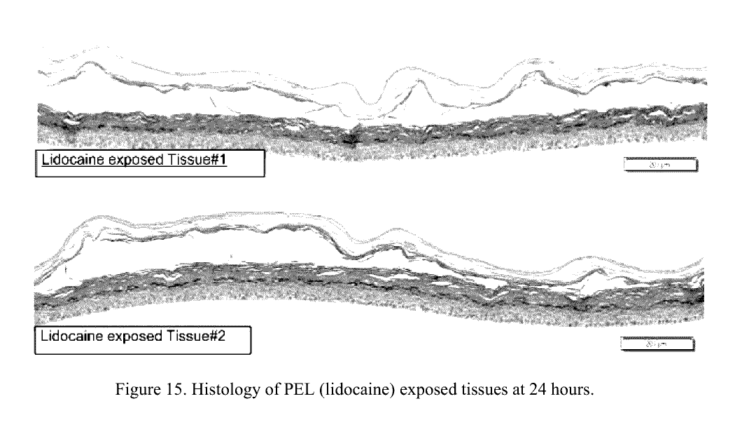

The concentrations of lidocaine in cell culture media are presented in Table 7. The result showed a four-fold increase in permeation at 24 hours after PEL exposure compared to the 2 and 4 hours time points. The four-fold increase in lidocaine permeation at 24 hours after PEL exposure corresponds to an approximate 75% absorption of the drug via the epidermal layer of the Epiderm™ model tissue. During the same 24 hours period of time, Epiderm™ tissues did not show any structural damage or significant changes in tissue morphology. (See FIG. 15).

The increased permeation of lidocaine at 24 hours post lidocaine exposure in the skin model is an interesting phenomenon, since lidocaine treated tissues showed no sign of tissue damage histologically. The permeation of lidocaine through the model skin and the stability of Epiderm™ tissues following the topical PEL exposure up to 24 hours suggest a potential use of prodosome encapsulated lidocaine for topical applications.

We claim:

1. A process for making one or more multilamellar clustoidal phospholipid structures, comprising the steps of:
(a) adding a naturally derived ionic mineral composition to water and mixing at high speed vortex to form ionically charged structured water;
(b) adding phosphatidylcholine of at least 70% purity to the ion-treated water composition by mixing in a high speed vortex to form a liposomal mixture;
(c) adding ethyl alcohol to the liposomal mixture by mixing in a high speed vortex to form the one or more multilamellar clustoidal phospholipid structures in water; and
(d) allowing the multilamellar clustoidal phospholipid structures in water to cool to ambient temperature.

2. A multilamellar clustoidal phospholipid vehicle for delivery of a cellular, subcellular, nutritional, nutritional, or pharmaceutical ingredient, comprising:
a solvent;
phosphatidylcholine of at least 70% purity; and
a naturally derived ionic mineral composition.

3. The multilamellar clustoidal phospholipid vehicle of claim 2, wherein the solvent is selected from the group consisting of water, an alcohol, and mixtures thereof.

4. The multilamellar clustoidal phospholipid vehicle of claim 2, wherein the multilamellar clustoidal phospholipid vehicle comprises one or more multilamellar clustoidal phospholipid structures.

5. The multilamellar clustoidal phospholipid vehicle of claim 2, wherein the naturally derived ionic mineral composition comprises one or more of sodium ion, magnesium ion, chloride ion, potassium ion, sulfate ion, boron ion, lithium ion, phosphorous ion, manganese ion, calcium ion, silicon ion, selenium ion, zinc ion, iodine ion, chromium ion, copper ion, molybdenum ion, or vanadium ion.

6. The multilamellar clustoidal phospholipid vehicle of claim 2, wherein the phosphatidylcholine is soy lecithin phospholipid.

7. The multilamellar clustoidal phospholipid vehicle of claim 2, wherein the phosphatidylcholine is impregnated and saturated with the naturally derived ionic mineral composition.

8. The multilamellar clustoidal phospholipid vehicle of claim 2, wherein the multilamellar clustoidal phospholipid vehicle is formulated in liquid dosage form.

9. The multilamellar clustoidal phospholipid vehicle of claim 2, wherein the multilamellar clustoidal phospholipid vehicle is formulated in solid dosage form.

10. The multilamellar clustoidal phospholipid vehicle of claim 2, wherein the naturally derived ionic mineral composition is present in an amount from about 0.1 percent to about 12 percent by weight of the vehicle.

11. The multilamellar clustoidal phospholipid vehicle of claim 2, wherein the phosphatidylcholine is present in an amount from about 2 percent to about 20 percent by weight of the vehicle.

12. The multilamellar clustoidal phospholipid vehicle of claim 2, wherein the solvent is water present in an amount from about 40 percent to about 80 percent by volume of the vehicle.

13. A formulation for delivery of an active ingredient, comprising:
the active ingredient encapsulated in a multilamellar clustoidal phospholipid vehicle, the multilamellar clustoidal phospholipid vehicle comprising:
a solvent;
phosphatidylcholine of at least 70% purity; and
a naturally derived ionic mineral composition.

14. The formulation of claim 13, wherein the active ingredient is selected from the group consisting of a cellular ingredient, a subcellular ingredient, a nutritional ingredient, a nutritional ingredient, a pharmaceutical ingredient, and mixtures thereof.

15. The formulation of claim 13, wherein the active ingredient is human platelets.

16. The formulation of claim 13, wherein the active ingredient is lidocaine.

17. The formulation of claim 13, wherein the active ingredient is one or more of multivitamins.

18. The formulation of claim 13, wherein the active ingredient is one or more of macro or trace minerals.

19. The formulation of claim 13, wherein the active ingredient is one or more of botanical nutrients or phytonutrients.

20. The formulation of claim 13, wherein the active ingredient is selected from the group consisting of NSAIDS, antibiotics, insulin, anesthetic agents, chemotherapeutic drugs, acne medications, vaccines, blood thinners, platelets, lidocaine, multivitamins, and mixtures thereof.

21. A method for delivering an active ingredient to an individual, comprising the steps of:
(a) providing a formulation comprising the active ingredient encapsulated in a multilamellar clustoidal phospholipid vehicle, the multilamellar clustoidal phospholipid vehicle comprising:
a solvent;
phosphatidylcholine of at least 70% purity; and
a naturally derived ionic mineral composition,
(b) administering the formulation to the individual in need thereof.

22. The method of claim 21, wherein the method of administration is selected from the group consisting of oral, intranasal, rectal, buccal, transmucosal, parenteral injection, transdermal, subcutaneous or intramuscular injections, subcutaneous needling, and nebulizer inhalation.

23. The method of claim 21, wherein the formulation is administered orally.

24. The method of claim 21, wherein the formulation is administered transdermally.

25. The method of claim 21, wherein the formulation is administered transmucosally.

26. The method of claim 21, wherein the active ingredient is selected from the group consisting of NSAIDS, antibiotics, insulin, anesthetic agents, chemotherapeutic drugs, acne medications, vaccines, blood thinners, platelets, lidocaine, multivitamins, and mixtures thereof.
